(19) United States
(12) Patent Application Publication
(10) Pub. No.: US 2025/0277801 A1
(43) Pub. Date: Sep. 4, 2025

(54) DERIVATISATION AGENT FOR LASER DESORPTION IONIZATION MASS SPECTROMETRY

(71) Applicant: Roche Diagnostics Operations, Inc., Indianapolis, IN (US)

(72) Inventors: Dirk Bernicke, Zinnowitz (DE); Robert Hahn, Muenchen (DE); Dieter Heindl, Muenchen (DE); Hannes Kuchelmeister, Muenchen (DE); Simon Loibl, Wolfratshausen (DE); Daniela Mazzier, Munich (DE); Giuseppe Prencipe, Koenigsdorf (DE); Martin Rempt, Penzberg (DE); Manuel Josef Seitz, Berg (DE); Christoph Zuth, Eglfing (DE)

(21) Appl. No.: 19/211,061

(22) Filed: May 16, 2025

Related U.S. Application Data
(63) Continuation of application No. PCT/EP2023/082387, filed on Nov. 20, 2023.

(30) Foreign Application Priority Data
Nov. 21, 2022 (EP) 22208585.4

(51) Int. Cl. G01N 33/74

(52) U.S. Cl. CPC G01N 33/743; G01N 2560/00

(57) ABSTRACT

In a first aspect, the invention relates to a derivatisation agent, preferably derivatisation agent for analytes intended to be analysed via LDI-MS, comprising a structural element of formula (I) C-L1-Z-(L2)ₚ-X, wherein C is a chromophore having an absorption maximum in the range of from 280 to 400 nm; Z is a charged unit comprising at least one permanently charged moiety; X is a reactive group; L1, L2 are each a linker unit; and p is either zero or 1.

A second aspect of the invention is related to a kit comprising the derivatisation agent according to the first aspect. In a third aspect, the invention is directed to a use of the derivatisation agent according to the first aspect for the mass spectrometric determination of an analyte molecule, wherein the mass spectrometric determination is LDI-MS. A fourth aspect of the invention relates to a conjugate of a derivatisation agent according to the first aspect and an analyte, wherein the conjugate has the structure of formula (II) C-L1-Z-(L2)ₚ-Xa-Ya-A, wherein C, L1, L2, p, Z and N are as defined in the context of the first aspect; Xa is a remainder of a reactive group X as defined in the context of the first aspect; A is the analyte and Ya is the remainder of a reactive group Y bound to the analyte A, which has reacted with the reactive group X of the derivatisation agent thus forming a covalent bound between Xa and Ya. A fifth aspect of the invention is related to a method for the mass spectrometric determination of an analyte molecule comprising the steps: (a) providing an analyte of interest; (b) providing a derivatisation agent comprising a structure of formula (I) as defined in the context of the first aspect; (c) reacting the analyte provided according to (a) with the derivatisation agent provided according to (b), whereby a conjugate of the analyte and the derivatisation agent is formed, and (d) subjecting the conjugate formed in (c) to a mass spectrometric analysis, wherein the mass spectrometric analysis is preferably LDI-MS.

In a first aspect, the invention relates to a derivatisation agent, preferably derivatisation agent for analytes intended to be analysed via LDI-MS, comprising a structural element of formula (I) C-L1-Z-(L2)ₚ-X, wherein C is a chromophore having an absorption maximum in the range of from 280 to 400 nm; Z is a charged unit comprising at least one permanently charged moiety; X is a reactive group; L1, L2 are each a linker unit; and p is either zero or 1.

A second aspect of the invention is related to a kit comprising the derivatisation agent according to the first aspect. In a third aspect, the invention is directed to a use of the derivatisation agent according to the first aspect for the mass spectrometric determination of an analyte molecule, wherein the mass spectrometric determination is LDI-MS. A fourth aspect of the invention relates to a conjugate of a derivatisation agent according to the first aspect and an analyte, wherein the conjugate has the structure of formula (II) C-L1-Z-(L2)ₚ-Xa-Ya-A, wherein C, L1, L2, p, Z and N are as defined in the context of the first aspect; Xa is a remainder of a reactive group X as defined in the context of the first aspect; A is the analyte and Ya is the remainder of a reactive group Y bound to the analyte A, which has reacted with the reactive group X of the derivatisation agent thus forming a covalent bound between Xa and Ya. A fifth aspect of the invention is related to a method for the mass spectrometric determination of an analyte molecule comprising the steps: (a) providing an analyte of interest; (b) providing a derivatisation agent comprising a structure of formula (I) as defined in the context of the first aspect; (c) reacting the analyte provided according to (a) with the derivatisation agent provided according to (b), whereby a conjugate of the analyte and the derivatisation agent is formed, and (d) subjecting the conjugate formed in (c) to a mass spectrometric analysis, wherein the mass spectrometric analysis is preferably LDI-MS.

### STATE OF THE ART

Mass spectrometry (MS) is a widely used technique for the qualitative and quantitative analysis of chemical substances ranging from small molecules to macromolecules. In general, it is a very sensitive and specific method, allowing even for the analysis of complex biological, e.g. environmental or clinical samples. However, for several analytes, especially if analyzed from complex biological matrices such as serum, sensitivity of the measurement remains an issue.

Often MS is combined with chromatographic techniques, particularly gas and liquid chromatography such as e.g. HPLC. Here, the analyted molecule of interest is separated chromatographically and is individually subjected to mass spectrometric analysis (Higashi et al. (2016) J. of Pharmaceutical and Biomedical Analysis 130 p. 181-190).

There is, however, still a need of increasing the sensitivity of MS analysis methods, particularly for the analysis of analytes that have a low abundance or when only little materials (such as biopsy tissues) are available.

In the art, several derivatisation reagents are known which aim to improve the sensitivity of the measurement for these analytes. Amongst others, reagents comprising charged units and neutral loss units which are combined in a single functional unit (e.g. WO 2011/091436 A1); other reagents for introducing neutral loss units and charged units are known, for example, from WO 2020/020850 A1. Other reagents comprising separate units are structurally relatively large which effects the general workflow of sample preparation and the MS measurement (Rahimoff et al. (2017) J. Am. Chem. Soc. 139(30), p. 10359-10364). Known derivatisation reagents are for example Cookson-type reagents, Amplifex Diene, Amplifex Keto, Girard T, Girard P. All of these bear disadvantages due to often insufficient labelling efficiencies, generation of structural isomers due to coupling chemistry, non-optimal ionization efficiencies, disadvantages for chromatographic separation after coupling, non-optima fragmentation behaviour due to many fragmentation pathways and need for high collision energies. There is thus an urgent need in the art for a derivatisation reagents which allows for a sensitive detection of analytes from complex biological matrices as well as exhibiting a chemical structure which does not negatively influence the MS measurement workflow. This is of particular importance in a random-access, high-throughput MS set up, wherein several different analytes exhibiting different chemical properties have to be measured in a short amount of time.

Chemical derivatisation of an analyte of interest can be used to enhance the detection sensitivity in mass spectrometry applications. In most cases, charged (i) or chargeable (ii) compounds are used to produce a (i) permanently charged or (ii) chargeable derivatized analyte in order to improve the mass spec response/sensitivity. Most of these reagents aim to enhance the ESI response, but they are not designed to generate a particular product ion by CID for MS/MS applications. To address this issue, several derivatisation reagents were developed carrying a structure suitable for MS/MS detection. In general numerous derivatisation reagents are available for liquid chromatography based mass spectrometry (i.e. LC-MS, LC-MS/MS; see reviews: J. Sep. Sci. 2016, 39, 102-114; Biomed. Chromatogr. 2011; 25: 1-10) and laser desorption based applications in combination with a suitable matrix (i.e. MALDI, see review: Trends in Analytical Chemistry 143 (2021) 116399; J Mass Spectrom. 2021; 56:e4731). However, the vast number of available derivatisation reagents do not possess a suitable chromophore to allow for efficient laser energy transfer in LDI applications and thereby are not suitable for high-sensitive LDI measurements. Consequently, these reagents need to be used in combination with a matrix, which causes an increased background, interferences and consequently a reduced sensitivity. Only few examples of derivatisation reagents are reported for matrix-free laser desorption applications, which are referred to so called “reactive matrices” (i.e. Anal. Chem. 2020, 92, 6224-6228; Chem Asian J. 2021, 16, 868-878 and Crit Rev Anal Chem. 2021 Dec. 30; 1-17) or “LDI labels” (i.e. Mass Spec Rev. 2019; 38:3-21; Scientific Reports|5:17853, ChemBioChem 2021, 22, 1430-1439;). The majority of these molecules offer a suitable chromophore, but are not permanently charged, which is disadvantageous in terms of high-sensitivity and are not designed for MS/MS applications. Rare examples of permanently charged and chromophoric derivatisation reagents are reported (i.e. International Journal of Mass Spectrometry 353 (2013) 54-59), but these reagents lack a suitable neutral loss site for and are consequently not suitable MS/MS applications. In most cases derivatisation reagent are small molecules, which generate, in case the analyte is also small molecule, again a low molecular weight derivatized analyte or in any case only a small mass shift after derivatisation. This can be problematic for several mass spec applications, since high sensitive measurements in the lower molecular weight range are often impeded by matrix-based MS-interferences in this range.

Thus, the problem underlying the present invention was the need for a permanently charged and chromophoric derivatisation reagent having a suitable neutral loss site.

### 1^{st} Aspect—Derivatisation Agent

In a first aspect, the problem was solved by a derivatisation agent, preferably a derivatisation agent for analytes intended to be analysed via laser desorption ionization mass spectrometry (LDI-MS), comprising a structural element of formula (I)

C-L1-Z-(L2)ₚ-X (I)
wherein
C is a chromophore having an absorption maximum in the range of from 280 to 400 nm;
Z is a charged unit comprising at least one permanently charged moiety;
X is a reactive group;
L1, L2 are each a linker unit; and
p is either zero or 1.

The derivatisation agent according to the present invention provides a solution for challenging high-sensitive measurements, preferably but not limited, in the field of laser desorption ionization (LDI)-based mass spectrometry (MS). The advantage of the derivatisation agent is an improved sensitivity for LDI-MS applications. The term “LDI-MS” comprises (MA)LDI-MS, preferably (MA)LDI-MS/MS and (SA)LDI-MS, preferably (SA)LDI-MS/MS, wherein “MALDI” (matrix-assisted laser desorption ionization) as well as “SALDI” (surface-assisted laser desorption ionization) are known and are also explained in more detail below in the section related to the third aspect of the invention. The derivatisation agent is distinguished by its chemical structural concept as well as its working principal from other reagents/solutions known in the art. On the one hand, the derivatisation agent carries a suitable chromophore, which enables an efficient energy transfer during LDI. On the other hand, the derivatisation agent adds a sufficient large molecular weight onto an analyte of interest, wherein the weight addition results in a sufficient mass shift beyond the high noise background of biological samples in the lower molecular weight region. Generally, it is known that such high molecular weight resulted in unfavorable ionization properties (poor ionization efficiency, multiple fragmentation processes . . . ), which is in the present case however circumvented by the presence of a permanent positive charge (Z unit in formula (I)). Therefore, the precursor ion (mother ion) can be detected with high sensitivity and, optionally, be selected for fragmentation in MS/MS applications. Efficient fragmentation is ensured by the specific combination of Z unit and N unit, which is in some embodiments outlined in more detail below a quaternary amine group neighboring a benzylic position, which enables a smooth and selective neutral loss fragmentation process. During MS/MS the conjugate of derivatisation reagent and analyte undergoes fragmentation by releasing a large fragment and the production ion (analyte of interest modified with a benzylic cation). This large mass shift is again highly advantageous, because it ensures a low background/interferences of this novel reagent class, since such specific and sensitive neutral loss pathways via large molecular weight loss are very uncommon (i.e. see Anal. Chem. 2014, 86, 21, 10724-10731).

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Percentages, concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “from 4 to 20%” should be interpreted to include not only the explicitly recited values of 4% to 20%, but to also include individual values and sub-ranges within the indicated range. Thus, included in this numerical range are individual values such as 4, 5, 6, 7, 8, 9, 10, . . . 18, 19, 20% and sub-ranges such as from 4-10%, 5-15%, 10-20%, etc. This same principle applies to ranges reciting minimal or maximal values. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “Mass Spectrometry” or “MS” relates to an analytical technology used to identify compounds by their mass. MS is a methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). The term “ionization” or “ionizing” refers to the process of generating an analyte ion having a net electrical charge equal to one or more electron units. Negative ions are those having a net negative charge of one or more electron units, while positive ions are those having a net positive charge of one or more electron units. The MS method may be performed either in “negative ion mode”, wherein negative ions are generated and detected, or in “positive ion mode” wherein positive ions are generated and detected.

“Tandem mass spectrometry” or “MS/MS” involves multiple steps of mass spectrometry selection, wherein fragmentation of the analyte occurs in between the stages. In a tandem mass spectrometer, ions are formed in the ion source and separated by mass-to-charge ratio in the first stage of mass spectrometry (MS1). Ions of a particular mass-to-charge ratio (precursor ions or parent ion) are selected and fragment ions (or daughter ions) are created by collision-induced dissociation, ion-molecule reaction, or photodissociation. The resulting ions are then separated and detected in a second stage of mass spectrometry (MS2).

While ionization sources such as electrospray ionization (ESI) and atmospheric pressure chemical ionization (APCI) are known, a reliable tool for ionization is Laser desorption ionization (LDI), for which common lasers are, for example, ultraviolet (355 nm) Nd:YAG lasers (neodymium-doped yttrium aluminum garnet; Nd:Y₃Al₅O₁₂).

Since a mass spectrometer separates and detects ions of slightly different masses, it easily distinguishes different isotopes of a given element. Mass spectrometry is thus, an important method for the accurate mass determination and characterization of analytes, including but not limited to low-molecular weight analytes, peptides, polypeptides or proteins. Its applications include the identification of proteins and their post-translational modifications, the elucidation of protein complexes, their subunits and functional interactions, as well as the global measurement of proteins in proteomics. De novo sequencing of peptides or proteins by mass spectrometry can typically be performed without prior knowledge of the amino acid sequence.

Mass spectrometric determination may be combined with additional analytical methods including chromatographic methods such as gas chromatography (GC), liquid chromatography (LC), particularly HPLC, and/or ion mobility-based separation techniques. In the context of the present disclosure, the term “analyte”, “analyte molecule”, or “analyte(s) of interest” are used interchangeably referring the chemical specis to be analysed via mass spectrometry. Chemical specis suitable to be analysed via mass spectrometry, i.e. analytes, can be any kind of molecule present in a living organism, include but are not limited to nucleic acid (e.g. DNA, mRNA, miRNA, rRNA etc.), amino acids, peptides, proteins (e.g. cell surface receptor, cytosolic protein etc.), metabolite or hormones (e.g. testosterone, estrogen, estradiol, etc.), fatty acids, lipids, carbohydrates, steroids, ketosteroids, secosteroids (e.g. Vitamin D), molecules characteristic of a certain modification of another molecule (e.g. sugar moieties or phosphoryl residues on proteins, methyl-residues on genomic DNA) or a substance that has been internalized by the organism (e.g. therapeutic drugs, drugs of abuse, toxin, etc.) or a metabolite of such a substance. Such analyte may serve as a biomarker. In the context of present invention, the term “biomarker” refers to a substance within a biological system that is used as an indicator of a biological state of said system. Most sample workflows in MS further include sample preparation and/or enrichment steps, wherein e.g. the analyte(s) of interest are separated from the matrix using e.g. gas or liquid chromatography.

In some preferred embodiments of the derivatisation agent, the absorption maximum of the chromophore C is an adsorption maximum determined by UV/VIS spectroscopy. Preferably, the absorption maximum of the chromophore C is in the range of from 290 to 380 nm, more preferably in the range of from 300 to 360 nm, more preferably in the range of from 305 to 330 nm.

In some preferred embodiments of the derivatisation agent, the chromophore C has a structure of formula (C)

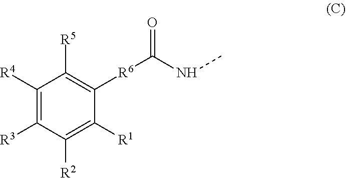

wherein R¹, R², R³, R⁴, R⁵ are independently selected from the group of hydrogen atom; hydroxyl group; NR^{x}R^{y} group, wherein R^{x} and R^{y} are independently a hydrogen atom or a C1 to C5 alkyl group; C1 to C5 alkyl, C5 to C10 (hetero)aryl and —O—C1 to C3 alkoxy group; and R⁶ is either none (i.e. there is a direct covalent single bond between the aromatic cycle and the C(═O) group) or is a —CR⁷═CR⁸— group, wherein R⁷ is a hydrogen atom or a C1 to C3 alkyl group and R⁸ is selected from the group consisting of hydrogen atom, C1 to C5 alkyl group and electron withdrawing group; and the dotted line represent a bond to the linker, which is preferably a single bond.

In some preferred embodiments of the derivatisation agent of formula (C), R¹, R², R³, R⁴, R⁵ are independently selected from the group of hydrogen atom, hydroxyl group, NR^{x}R^{y} group, wherein R^{x} and R^{y} are independently a hydrogen atom or a C1 to C5 alkyl group, and —O—C1 to C3 alkoxy group. More preferably, R¹, R², R³, R⁴, R⁵ are independently selected from the group of hydrogen atom, hydroxyl group, NR^{x}R^{y} group, wherein R^{x} and R^{y} are independently a hydrogen atom or a C1 to C5 alkyl group and —O—C1 to C3 alkoxy group, with the condition that at least one of R¹, R², R³, R⁴, R⁵ is either a hydroxyl group or a —O—C1 to C3 alkoxy group. The electron withdrawing group of R⁸ is preferably selected from cyano group, nitro group, carboxyl group, halogen atom (preferably fluoro atom, chloro atom, bromine atom or iodine atom), and aryl group, wherein “aryl” is preferably selected from the group of C5 to C10 (hetero)aryl group, more preferably phenyl, wherein the electron withdrawing group of R⁸ is more preferably a cyano (C≡N group).

In some preferred embodiments of the derivatisation agent, the chromophore C has a structure of formula (C1), (C2) or (C3):

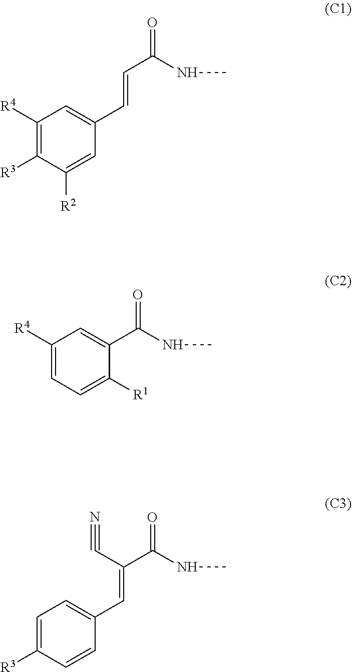

wherein R¹, R², R³, R⁴, are independently selected from the group of hydrogen atom, hydroxyl group NR^{x}R^{y} group, wherein R^{x} and R^{y} are independently a hydrogen atom or a C1 to C5 alkyl group, C1 to C5 alkyl, C5 to C10 (hetero)aryl and —O—C1 to C3 alkoxy group.

Also for (C1), (C2) and (C3), it applies that in some preferred embodiments, R¹, R², R³, R⁴, R⁵ are independently selected from the group of hydrogen atom, hydroxyl group, NR^{x}R^{y} group, wherein R^{x} and R^{y} are independently a hydrogen atom or a C1 to C5 alkyl group, and —O—C1 to C3 alkoxy group. More preferably, R¹, R², R³, R⁴, R⁵ are independently selected from the group of hydrogen atom, hydroxyl group, NR^{x}R^{y} group, wherein R^{x} and R^{y} are independently a hydrogen atom or a C1 to C5 alkyl group, and —O—C1 to C3 alkoxy group, with the condition that at least one of R¹, R², R³, R⁴, R⁵ is either a hydroxyl group or a —O—C1 to C3 alkoxy group. The electron withdrawing group of R⁸ is preferably selected from cyano group, nitro group, carboxyl group, halogen atom (preferably fluoro atom, chloro atom, bromine atom or iodine atom), and aryl group, wherein “aryl” is preferably selected from the group of C5 to C10 (hetero)aryl group, more preferably phenyl, wherein the electron withdrawing group of R⁸ is more preferably a cyano (C≡N group).

In some preferred embodiments of the derivatisation agent, the chromophore C has a structure of formula (C1), wherein R¹ and R⁴ are both a methoxy group and R³ is a hydroxyl group (C1 with these residues R², R³ and R⁴ being the remainder of sinapinic acid). In some preferred embodiments of the derivatisation agent, the chromophore C has a structure of formula (C2), wherein R² and R⁴ are both a hydroxyl group (C2 with these residues R², R⁴ being the remainder of 2,5-dihydroxy benzoic acid). In some preferred embodiments of the derivatisation agent, the chromophore C has a structure of formula (C3), wherein R³ is a hydroxyl group (C3 with this residue R³ being the remainder of alpha-cyano-4-hydroxy-cinamix acid).

In some preferred embodiments of the derivatisation agent, the linker L1 is selected from the group consisting of (C1-C5 alkylene-O—)ₘ group with m being an integer in the range of from 1 to 10, C1 to C20 alkylene group, C1 to C20 alkylene group-heteroaryl group and (C1-C5 alkylene)-O—(C1-C5 alkylene) group, optionally connected to or intersected by a unit selected from the group consisting of (hetero)aryl group, N₂, NO, NO₂, S₂, SO, SO₂, CO, and CO₂, said unit being preferably, if present, a heteroaryl group, more preferably from triazol, phenyltriazol, tetrazol and phenyltetrazol. A “heteroaryl” is preferably a C1 to C10 heteroaryl with at least one heteroatom as part of the ring structure, wherein the at least one heteroatom is preferably selected from N, O and S, more preferably the heteroaryl is selected from the group consisting of triazole, tetrazole, tetrazine, oxadiazole, thiadiazole and any hydrogenated derivative thereof, more preferably from the group consisting of 1,2,3-triazole, 1,2,4-triazole, 1,4,5-triazole, 3,4,5-triazole, 1,2,3,4-tetrazole, 2,3,4,5-tetrazole, 2,3,5,6 tetrazole and 1,2,4,5 tetrazine. In some preferred embodiments of the derivatisation agent, the linker L1 has a structure (L1a)_{q}-(L1b)ᵣ-(L1c)ₛ, wherein q, r, s are each zero or 1, with the condition that at least one of q, r, s is 1; L1a being selected from the group consisting of (C1-C5 alkylene-O—)ₘ group with m being an integer in the range of from 1 to 10, C1 to C10 alkylene group; L1b being a unit selected from the group consisting of N₂, NO, NO₂, S₂, SO, SO₂, CO, CO₂, triazol, phenyltriazol, tetrazol and phenyltetrazol, wherein the unit is preferably a triazol or a tetrazol, more preferably the unit N is a triazol, more preferably the unit N is a 1,2,3 triazol ring, which is bound to the linker L1a, if present, via a single bond at position 1 (N atom) of the triazol ring and bound to the Linker L1c, if present, via a single bond at position 4 (C atom) of the triazol ring; L1c, if present, being selected from the group consisting of (C1-C5 alkylene-O—)ₘ group with m being an integer in the range of from 1 to 10, C1 to C10 alkylene group and (C1-C5 alkylene)-O—(C1-C5 alkylene) group. In some preferred embodiments of the derivatisation agent, the linker L1 has a structure

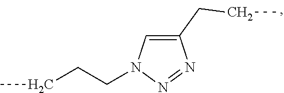

wherein the dotted lines indicate the bonds to (C) and (Z) respectively.

In some preferred embodiments of the derivatisation agent, the charged unit Z is positively or negatively charged, preferably positively charged. In some preferred embodiments of the derivatisation agent, the charged unit Z is positively charged and is preferably a tetraalkyl ammonium group, more preferably a —CH₂N⁺(CH₃)₂CH₂— group. In some preferred alternative embodiments of the derivatisation agent, the charged unit Z is negatively charged, wherein the negatively charged unit Z is preferably selected from the group consisting of a phosphate, sulphate, sulphonate and carboxylate.

In some preferred embodiments of the derivatisation agent, the linker L2 comprises 1 to 10 C atoms and optionally one or more heteroatom(s). Preferably, the linker L2 is a C1-C5-alkylene-C5 to C10 aromatic ring, which preferably bears a C(═O) unit as substituent of the aromatic ring, the linker L2 more preferably being a C1-C3-alkylene-C6 aromatic ring, which preferably bears a C(═O) unit as substituent of the aromatic ring, wherein the C6 aromatic ring is more preferably a substituted or unsubstituted benzene ring, more preferably benzene ring, which bears a C(═O) group at the position of the benzene ring which is para with respect to the position where the C1-C3-alkylene is bound and which more preferably bears no further substituents.

In some preferred embodiments of the derivatisation agent, the reactive group X is selected from the group consisting of carbonyl reactive unit, diene reactive unit, hydroxyl reactive unit, amino reactive unit, imine reactive unit, thiol reactive unit, diol reactive unit, phenol reactive unit, epoxide reactive unit, disulfide reactive unit, and azido reactive unit.

In some preferred embodiments of the derivatisation agent, the reactive unit X is a carbonyl reactive unit, which is capable of reacting with any type of molecule having a carbonyl group. The carbonyl reactive unit is preferably selected from the group consisting of carboxyl reactive unit, keto reactive unit, aldehyde reactive unit, anhydride reactive unit, carbonyl ester reactive unit, and imide reactive unit. In some preferred embodiments of the derivatisation agent, the carbonyl-reactive unit may have either a super-nucleophilic N atom strengthened by the a-effect through an adjacent O or N atom NH₂—N/O or a dithiol molecule.

In some preferred embodiments of the derivatisation agent, the carbonyl-reactive unit is selected from the group:
(i) a hydrazine unit, e.g. a H₂N—NH—, or H₂N—NR^{a}— unit, wherein R^{a} is aryl, aryl containing 1 or more heteroatoms or C1-4 alkyl, particularly C1 or C2 alkyl, optionally substituted e.g. with halo, hydroxyl, and/or C1-3 alkoxy,
(ii) a hydrazide unit, in particular a carbo-hydrazide or sulfo-hydrazide unit, in particular a H₂N—NH—C(O)—, or H₂N—NR^{b}—C(O)— unit,
   wherein R^{b} is aryl, aryl containing 1 or more heteroatoms or C1-4 alkyl, particularly Ci or C2 alkyl, optionally substituted e.g. with halo, hydroxyl, and/or C1-3 alkoxy,
(iii) a hydroxylamino unit, e.g. a H₂N—O— unit, and
(iv) a dithiol unit, particularly a 1,2-dithiol or 1,3-dithiol unit.

In some preferred embodiments of the derivatisation agent, wherein the carbonyl reactive unit is a carboxyl reactive unit, the carboxyl reactive units reacts with carboxyl groups on an analyte molecule. In embodiment of the first aspect of the present invention, the carboxyl reactive unit is selected from the group consisting of a diazo unit, an alkylhalide, amine, and hydrazine unit.

In some preferred embodiments of the derivatisation agent, the reactive unit X is a diene reactive unit, which is capable of reacting with an analyte comprising a diene group. In some preferred embodiments of the derivatisation agent, the diene reactive unit is selected from the group consisting of Cookson-type reagents, e.g. 1,2,4-triazolin-3,5-diones, which are capable to act as a dienophile.

In some preferred embodiments of the derivatisation agent, the reactive unit X is a hydroxyl reactive unit, which is capable of reacting with an analyte comprising a hydroxyl group. In some preferred embodiments of the derivatisation agent, the hydroxyl reactive units is selected from the group consisting of sulfonylchlorides, activated carboxylic esters (NHS, or imidazolide), and fluoro aromates/heteroaromates capable for nucleophilic substitution of the fluorine (T. Higashi J Steroid Biochem Mol Biol. 2016 September; 162:57-69). In some preferred embodiments of the derivatisation agent, the reactive unit X is a diol reactive unit, which reacts with an diol group on an analyte molecule. In some preferred embodiments of the derivatisation agent, wherein the reactive unit is a 1,2 diol reactive unit, the 1,2 diol reactive unit comprises boronic acid. In further embodiments, diols can be oxidised to the respective ketones or aldehydes and then reacted with ketone/aldehyde-reactive units X. In some preferred embodiments of the derivatisation agent, the amino reactive unit reacts with amino groups on an analyte molecule. In some preferred embodiments of the derivatisation agent, the amino-reactive unit is selected from the group consisting of active ester group such as N-hydroxy succinimide (NHS) ester or sulfo-NHS ester, pentafluoro phenyl ester, cabonylimidazole ester, quadratic acid esters, a hydroxybenzotriazole (HOBt) ester, 1-hydroxy-7-azabenzotriazole (HO At) ester, and a sulfonylchloride unit.

In some preferred embodiments of the derivatisation agent, the thiol reactive unit reacts with an thiol group on an analyte molecule. In some preferred embodiments of the derivatisation agent, the thiole reactive unit is selected from the group consisting of haloacetyl group, in particular selected from the group consisting of Br/I—CH₂—C(═O)— unit, acrylamide/ester unit, unsaturated imide unit such as maleimide, methylsulfonyl phenyloxadiazole and sulfonylchloride unit.

In some preferred embodiments of the derivatisation agent, the phenol reactive unit reacts with phenol groups on an analyte molecule. In some preferred embodiments of the derivatisation agent, the phenol-reactive unit is selected from the group consisting of active ester unit such as N-hydroxy succinimide (NHS) ester or sulfo-NHS ester, pentafluoro phenyl ester, carbonylimidazole ester, quadratic acid esters, a hydroxybenzotriazole (HOBt) ester, 1-hydroxy-7-azabenzotriazole (HO At) ester, and a sulfonylchloride unit. Phenol groups present on an analyte molecule can be reacted with triazole dione via a reaction (H. Ban et al J. Am. Chem. Soc., 2010, 132 (5), pp 1523-1525) or by diazotization or alternatively by ortho nitration followed by reduction to an amine which could then be reacted with an amine reactive reagent.

In some preferred embodiments of the derivatisation agent, the reactive unit X is a epoxide reactive unit, which is capable of reacting with an analyte comprising a epoxide group. In some preferred embodiments of the derivatisation agent, the epoxide reactive unit is selected from the group consisting of amino, thiol, super-nucleophilic N atom strengthened by the a-effect through an adjacent O or N atom NH₂—N/O molecule.

In some preferred embodiments of the derivatisation agent, the epoxide reactive unit is selected from the group:
(i) a hydrazine unit, e.g. a H₂N—NH—, or H₂N—NR^{a}— unit, wherein R^{a} is aryl, aryl containing 1 or more heteroatoms or C1-4 alkyl, particularly Ci or C2 alkyl, optionally substituted e.g. with halo, hydroxyl, and/or C1-3 alkoxy,
(ii) a hydrazide unit, in particular a carbo-hydrazide or sulfo-hydrazide unit, in particular a H₂N—NH—C(O)—, or H2 N—NR^{b}—C(O)— unit,
   wherein R^{b} is aryl, aryl containing 1 or more heteroatoms or Ci-4 alkyl, particularly Ci or C2 alkyl, optionally substituted e.g. with halo, hydroxyl, and/or C1-3 alkoxy, and
(iii) a hydroxylamino unit, e.g. a H₂N—O— unit.

In some preferred embodiments of the derivatisation agent, the reactive unit X is a disulfide reactive unit, which is capable of reacting with an analyte comprising a disulfide group. In some preferred embodiments of the derivatisation agent, the disulfide reactive unit is selected from the group consisting of thiol. In further embodiments, disulfide group can be reduced to the respective thiol group and then reacted with thiol reactive units X.

In some preferred embodiments of the derivatisation agent, the reactive unit X is a azido reactive unit which reacts with azido groups on an analyte molecule. In some preferred embodiments of the derivatisation agent, the azido-reactive unit reacts with azido groups through azide-alkyne cycloaddition. In some preferred embodiments of the derivatisation agent, the azido-reactive unit is selected from the group consisting of alkyne (alkyl or aryl), linear alkyne or cyclic alkyne. The reaction between the azido and the alkyne can proceed with or without the use of a catalyst. In further embodiments of the first aspect of the present invention the azido group can be reduced to the respective amino group and then reacted with amino reactive units X.

### 2^{nd} Aspect—Kit

A second aspect of the present invention is directed to a kit comprising the derivatisation agent according to the first aspect. All details, embodiment, and preferred embodiments as disclosed above in the section related to the first aspect apply also for the kit of the second aspect.

A “kit” is any manufacture (e.g. a package or container) comprising at least one reagent, e.g., a medicament for treatment of a disorder, or a probe for specifically detecting a biomarker gene or protein of the invention. The kit is preferably promoted, distributed, or sold as a unit for performing the use and/or the method of the present invention as described below in the sections related to the third and the fourth aspects of the invention.

Typically, a kit may further comprise carrier means being compartmentalized to receive in close confinement one or more container means such as vials, tubes, and the like In particular, each of the container means comprises one of the separate elements to be used in the method of the first aspect. Kits may further comprise one or more other containers comprising further materials including but not limited to buffers, diluents, filters, needles, syringes, and package inserts with instructions for use. A label may be present on the container to indicate that the composition is used for a specific application, and may also indicate directions for either in vivo or in vitro use. The computer program code may be provided on a data storage medium or device such as a optical storage medium (e.g., a Compact Disc) or directly on a computer or data processing device. Moreover, the kit may, comprise standard amounts for the biomarkers as described elsewhere herein for calibration purposes.

A “package insert” is used to refer to instructions customarily included in commercial packages of therapeutic products or medicaments, that contain information about the indications, usage, dosage, administration, contraindications, other therapeutic products to be combined with the packaged product, and/or warnings concerning the use of such therapeutic products or medicaments, etc.

### 3^{rd} Aspect—Use of a Derivatisation Agent for the Mass Spectrometric Determination of an Analyte Molecule

In a third aspect, the invention is directed to a use of a derivatisation agent according to the first aspect for the mass spectrometric determination of an analyte molecule, wherein the mass spectrometric determination is laser desorption ionization mass spectrometry (LDI-MS), preferably is (MA)LDI-MS, more preferably (MA)LDI-MS/MS, or (SA)LDI-MS, more preferably (SA)LDI-MS/MS. All details, embodiment, and preferred embodiments as disclosed above in the sections related to the first aspect and the second aspect apply also for the use of the third aspect. “MS” and “MS/MS” have a meaning as explained above in the section related to the first aspect.

The term “LDI” is the common abbreviation for laser desorption ionization, which, in applications where combined with a suitable matrix, is called matrix-assisted laser desorption ionization “MALDI”. Matrix materials and supports such as metal, especially steel, plates are known to the skilled person, the same applies for the conditions to be applied. The matrix typically consists of crystallized molecules, of which the three most commonly used are sinapinic acid, alpha-cyano-4-hydroxycinnamic acid (alpha-CHCA, alpha-cyano or alpha-matrix) and 2,5-dihydroxybenzoic acid DHB). MALDI techniques typically employ the use of UV lasers such as nitrogen lasers (337 nm) and frequency-tripled and quadrupled Nd:YAG lasers (355 nm and 266 nm respectively). Infrared laser wavelengths used for infrared MALDI include the 2.94 μm Er:YAG laser, mid-IR optical parametric oscillator, and 10.6 μm carbon dioxide laser.

Surface-assisted laser desorption ionization “SALDI” is a soft laser desorption technique without using matrix molecules but rather using a medium that absorbs energy from a laser and then transfers the energy to the target sample, wherein the active surface of the specific substrate plays the decisive role. Important substrates are solid surfaces of porous silicon. The porous silicon represented the first matrix-free SALDI surface analysis allowing for facile detection of intact molecular ions. SA multitude of different surfaces are known to work as SALDI substrates. Based on the elemental composition, the majority of the SALDI substrates reported in the literature can commonly be classified into three main types: carbon-based, semiconductor-based and metallic-based. The SALDI process using inorganic matrices for the preparations is described in several works, for example, in Law et al. (Anal. Bioanal. Chem. 2011, 399, 2597, DOI 10.1007/s00216-010-4063-3). Preferably, in the context of the present invention, a SALDI-MS target plate based on a steel plate with a functional amorphous a-C:H:Si:X (X=heteroatom modified) plasma activated chemical vapor deposited (PACVD) surface coating as top layer is used.

The laser-desorption ionization mass spectrometric (LDI-MS) measurements are preferably performed in positive ion mode, using utilizing a Nd:YAG-laser wavelength of 355 nm. The laser repetition rates of the LDI-MS system are set as appropriate for MALDI experiments and as appropriate for SALDI experiments respectively. Further parameters of the measurements, such as moving patterns, movement speed, frequency, acquisition time, mass spectral scan time, laser intensity or voltage settings are selected as appropriate and known to a person skilled in the art, who is also familiar with the required software based data analysis tools.

### 4^{th} Aspect—Conjugate

A fourth aspect of the invention relates to a conjugate of a derivatisation agent according to the first aspect of the invention and an analyte, wherein the conjugate has the structure of formula (II)

C-L1-Z-(L2)ₚ-Xa-Ya-A (II)
wherein C, L1, L2, p, Z and N are as defined in the section related to the first aspect; Xa is a remainder of a reactive group X as defined in the section related to the first aspect; A is the analyte and Ya is the remainder of a reactive group Y bound to the analyte A, which has reacted with the reactive group X of the derivatisation agent thus forming a covalent bound between Xa and Ya. All details, embodiments and preferred embodiments as described above in the sections related to the first, the second and the third aspect of the invention apply also for the conjugate of the fourth aspect, especially all details, embodiments and preferred embodiments as described above in the sections related to the first aspect apply also here.

In some preferred embodiments of the conjugate, the analyte is selected from the group consisting of nucleic acid (preferably selected from DNA, mRNA, miRNA, and rRNA), amino acid, peptide, protein (preferably cell surface receptor or cytosolic protein), metabolite, hormone (preferably selected from testosterone, estrogen and estradiol), fatty acid, lipid, carbohydrate, steroid, ketosteroid, secosteroid (Vitamin D), molecule characteristic of a certain modification of another molecule (preferably selected from sugar moiety, phosphoryl residue on a protein, methyl-residue on genomic DNA), substance that has been internalized by the organism (preferably selected from therapeutic drug, drug of abuse, toxin) and a metabolite of such a substance.

Analytes may be present in a sample of interest, e.g. a biological or clinical sample. The term “sample” or “sample of interest” are used interchangeably herein, referring to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. Examples of samples include but are not limited to fluid samples such as blood, serum, plasma, synovial fluid, spinal fluid, urine, saliva, and lymphatic fluid, or solid samples such as dried blood spots and tissue extracts. Further examples of samples are cell cultures or tissue cultures.

In the context of the present disclosure, the sample may be derived from an “individual” or “subject”. Typically, the subject is a mammal. Mammals include, but are not limited to, domesticated animals (e.g., cows, sheep, cats, dogs, and horses), primates (e.g., humans and non-human primates such as monkeys), rabbits, and rodents (e.g., mice and rats).

Before being analysed via Mass Spectrometry, a sample may be pre-treated in a sample-and/or analyte specific manner. In the context of the present disclosure, the term “pre-treatment” refers to any measures required to allow for the subsequent analysis of a desired analyte via Mass Spectrometry. Pre-treatment measures typically include but are not limited to the elution of solid samples (e.g. elution of dried blood spots), addition of hemolizing reagent (HR) to whole blood samples, and the addition of enzymatic reagents to urine samples. Also the addition of internal standards (ISTD) is considered as pre-treatment of the sample.

The term “hemolysis reagent (HR)” refers to reagents which lyse cells present in a sample, in the context of this invention hemolysis reagents in particular refer to reagents which lyse the cell present in a blood sample including but not limited to the erythrocytes present in whole blood samples. A well known hemolysis reagent is water (H2O). Further examples of hemolysis reagents include but are not limited to deionized water, liquids with high osmolarity (e.g. 8M urea), ionic liquids, and different detergents.

Typically, an internal standard (ISTD) is a known amount of a substance which exhibits similar properties as the analyte of interest when subjected to the mass spectrometric detection worklflow (i.e. including any pre-treatment, enrichment and actual detection step). Although the ISTD exhibits similar properties as the analyte of interest, it is still clearly distinguishable from the analyte of interest. Exemplified, during chromatographic separation, such as gas or liquid chromatography, the ISTD has about the same retention time as the analyte of interest from the sample. Thus, both the analyte and the ISTD enter the mass spectrometer at the same time. The ISTD however, exhibits a different molecular mass than the analyte of interest from the sample. This allows a mass spectrometric distinction between ions from the ISTD and ions from the analyte by means of their different mass/charge (m/z) ratios. Both are subject to fragmentation and provide daughter ions. These daughter ions can be distinguished by means of their m/z ratios from each other and from the respective parent ions. Consequently, a separate determination and quantification of the signals from the ISTD and the analyte can be performed. Since the ISTD has been added in known amounts, the signal intensity of the analyte from the sample can be attributed to a specific quantitative amount of the analyte. Thus, the addition of an ISTD allows for a relative comparison of the amount of analyte detected, and enables unambiguous identification and quantification of the analyte(s) of interest present in the sample when the analyte(s) reach the mass spectrometer. Typically, but not necessarily, the ISTD is an isotopically labeled variant (comprising e.g. 2 H, 13 C, or 15 N etc. label) of the analyte of interest.

In addition to the pre-treatment, the sample may also be subjected to one or more enrichment steps. In the context of the present disclosure, the term “first enrichment process” or “first enrichment workflow” refers to an enrichment process which occurs subsequent to the pre-treatment of the sample and provides a sample comprising an enriched analyte relative to the initial sample. The first enrichment workflow may comprise chemical precipitation (e.g. using acetonitrile) or the use of a solid phase. Suitable solid phases include but are not limited to Solid Phase Extraction (SPE) cartridges, and beads. Beads may be non-magnetic, magnetic, or paramagnetic. Beads may be coated differently to be specific for the analyte of interest. The coating may differ depending on the use intended, i.e. on the intended capture molecule. It is well-known to the skilled person which coating is suitable for which analyte. The beads may be made of various different materials. The beads may have various sizes and comprise a surface with or without pores.

In the context of the present disclosure the term “second enrichment process” or “second enrichment workflow” refers to an enrichment process which occurs subsequent to the pre-treatment and the first enrichment process of the sample and provides a sample comprising an enriched analyte relative to the initial sample and the sample after the first enrichment process.

In some preferred embodiments of the conjugate, the reactive group Y is selected from the group consisting of carbonyl group, diene group, hydroxyl group, amine group, imine group, thiol group, diol group, phenolic group, expoxid group, disulfide group, and azide group.

In some preferred embodiments of the conjugate, the analyte molecule, before being reacted with the derivatisation agent, comprises a functional group selected from the group above, wherein each of the functional groups indicated in this group is capable of forming a covalent bond with reactive unit X of the derivatisation agent. Further, it is also contemplated within the scope of the present invention that a functional group present on an analyte molecule would be first converted into another group that is more readily available for reaction with reactive unit X of the derivatisation agent.

In some embodiments, the analyte molecule, before being reacted with the derivatisation agent, comprises a carbonyl group as functional group which is selected from the group consisting of a carboxylic acid group, aldehyde group, keto group, a masked aldehyde, masked keto group, ester group, amide group, and anhydride group. In embodiments where the carbonyl group is an amide group, the skilled person is well-aware that the amide group as such is a stable group, but that it can be hydrolized to convert the amide group into an carboxylic acid group and an amino group. Hydrolysis of the amide group may be achieved via acid/base catalysed reaction or by enzymatic process either of which is well-known to the skilled person. In embodiments where the carbonyl group is a masked aldehyde group or a masked keto group, the respective group is either a hemiacetal group or acetal group, in particular a cyclic hemiacetal group or acetal group. In some embodiments, the acetal group is converted into an aldehyde or keto group before reaction with the derivatisation agent. In some embodiments, the carbonyl group is a keto group. The keto group may be transferred into an intermediate imine group before reacting with the reactive unit of the derivatisation agent. In some embodiments, the analyte molecule comprising one or more keto groups is preferably a ketosteroid. In particular embodiments, the ketosteroid is selected from the group consisting of testosterone, epitestosterone, dihydrotestosterone (DHT), desoxymethyltestosterone (DMT), tetrahydrogestrinone (THG), aldosterone, estrone, 4-hydroxyestrone, 2-methoxyestrone, 2-hydroxyestrone, 16-ketoestradiol, 16 alpha-hydroxyestrone, 2-hydroxyestrone-3-methylether, prednisone, prednisolone, pregnenolone, progesterone, DHEA (dehydroepiandrosterone), 17-OH pregnenolone, 17-OH progesterone, 17-OH progesterone, androsterone, epiandrosterone, and delta 4 androstenedione) 11-desoxycortisol corticosterone, 21-deoxycortisol, 11-deoxycorticosterone, allopregnanolone, and aldosterone.

In some embodiments, the carbonyl group is a carboxyl group. The carboxyl group reacts directly with the derivatisation agent or it is converted into an activated ester group before reaction with the derivatisation agent. In some embodiments, the analyte molecule comprising one or more carboxyl groups is selected from the group consisting of D8-Tetrahydrocannabinol-acid, Benzoylecgonin, Salicylic acid, 2-hydroxybenzoic acid, Gabapentin, Pregabalin, Valproic acid, Vancomycin, Methotrexat, Mycophenolic acid, Montelukast, Repaglinide, Furosemide, Telmisartan, Gemfibrozil, Diclorofenac, Ibuprofen, Indomethacin, Zomepirac, Isoxepac, and Penicilin. In some embodiments, the analyte molecule comprising one or more carboxyl groups is preferably an amino acid preferably selected from the group consisting of arginine, lysine, aspartic acid, glutamic acid, glutamine, asparagine, histidine, serine, threonine, tyrosine, cysteine, tryptophan, alanine, isoleucine, leucine, methionine, phenyalanine, valine, proline, and glycine.

In some embodiments, the carbonyl group is an aldehyde group. The aldehyde group may be transferred into an intermediate imine group before reacting with the reactive unit of the derivatisation agent. In some embodiments, the analyte molecule comprising one or more aldehyde groups is preferably selected from the group consisting of Pyridoxal, N-Acetyl-D-glucosamine, Alcaftadine, Streptomycin, Josamycin.

In some embodiments, the carbonyl group is an carbonyl ester group. The analyte molecule comprising one or more ester groups is preferably selected from the group consisting of Cocaine, Heroin, Ritalin, Aceclofenac, Acetycholine, Amcinonide, Amiloxate, amylocaine, Anileridine, Aranidipine, and Artesunate, Pethidine.

In some embodiments, the carbonyl group is an anhydride group. The analyte molecule comprising one or more anhydride groups is preferably selected from the group consisting of Cantharidin, Succinic Anhydride, Trimellitic Anhydride, and Maleic Anhydride.

In some embodiments, the analyte molecule comprises one or more diene groups, in particular to conjugated diene groups, as functional group. The analyte molecule comprising one or more diene groups is preferably a secosteroid. In some embodiments, the secosteroid is selected from the group consisting of Cholecaleiferol (Vitamin D3), Ergocalciferol (Vitamin D2), Calcidiol, Calcitriol, Tachysterol, Lumisterol und Tacalcitol. In particular, the secosteroid is Vitamin D, in particular Vitamin D2 or D3 or derivates thereof. In particular embodiments, the secosteroid is selected from the group consisting of Vitamin D2, Vitamin D3, 25-Hydroxy Vitamin D2, 25-Hydroxy Vitamin D3, 3-Epi-25-Hydroxy Vitamin D2, 3-Epi-25-Hydroxy Vitamin D3, 1,25-Dihydroxy Vitamin D2, 1,25-Dihydroxy Vitamin D3, 24,25-Dihydroxy Vitamin D2, and 24,25-Dihydroxy Vitamin D3, Vitamin A, Tretinoin, Isotretinoin, Alitretinoin, Natamycin, Sirolimus, Amphotericin B, Nystatin, Everolimus, Temsirolimus, Fidaxomicin.

In some embodiments, the analyte molecule comprises one or more hydroxyl group as functional group. The analyte molecule then preferably comprises a single hydroxyl group or two hydroxyl groups. In embodiments wherein more than one hydroxyl group is present, the two hydroxyl groups may be positioned adjacent to each other (1,2 diol) or may be separated by 1, 2, or 3 C-atoms (1,3-diol, 1,4-diol, 1,5-diol, respectively). In particular embodiments, the analyte molecule comprises an 1,2 diol group. In embodiments, wherein only one hydroxyl group is present, said analyte is preferably selected from the group consisting of primary alcohol, secondary alcohol and tertiary alcohol. In some embodiments, wherein the analyte molecule comprises one or more hydroxyl groups, the analyte is preferably selected from the group consisting of Benzyl alcohol, Menthol, L-Camitine, Pyridoxine, Metronidazole, Isosorbide mononitrate, Guaifenesin, Clavulanate, Migitol, Zalcitabine, Isoprenaline, Aciclovir, Methocarbamol, Tramadol, Venlafaxine, Atropine, Clofedanol, alpha-Hydroxyalprazolam, Alpha-Fly droxytriazolam, Forazepam, Oxazepam, Tamazepam, Ethylglucuronide, Ethylmorphine, Morphine, Morphine-3-glucuronide, Buprenorphine, Codeine, Dihydrocodeine, p-Hydroxypropoxyphene, O-desmethyltramadol, Dihydroquinidine, Quinidine. In some embodiments, wherein the analyte molecule comprises more than one hydroxyl groups, the analyte is preferably selected from the group consisting of Vitamin C, Glucosamine, Mannitol, Tetrahydrobiopterin, Cytarabine, Azacitidine, Ribavirin, Floxuridine, Gemcitadine, Streptozocin, Adenosine, Vibarabine, Cladribine, Estriol, Trifluridine, Clofarabine, Nadolol, Zanamivir, Factulose, Adenosine monophosphate, Idoxuridine, Regadenoson, Fincomycin, Clindamycin, Canaglifozin, Tobramycin, Netilmicin, Kanamycin, Ticagrelor, Epirubicin, Doxorubicin, Arbekacin, Steptomycin, Quabain, Amikacin, Neomycin, Framycetin, Paromomycin, Erythromycin, Clarithromycin, Azithromycin, Vindesine, Digitoxin, Digoxin, Metrizamide, Acetyldigitoxin, Deslanoside, Fludaradine, Clofarabine, Gemcitabine, Cytarabine, Capecitabine, Vidarabine, Trifluridine, Idoxuridine, and Plicamycin.

In some embodiments, the analyte molecule comprises one or more thiol group (including but not limited to alkyl-thiol and thiol aryl groups) as functional group. The analyte molecule comprising one or more thiol groups is preferably selected from the group consisting of Thiomandelic acid, DL-Captopril, DL-Thiorphan, N-Acetylcysteine, D-Penicillamine, Glutathione, L-Cysteine, Zefenoprilat, Tiopronin, Dimercaprol, Succimer.

In some embodiments, the analyte molecule comprises one or more disulfide group as functional group. The analyte molecule comprising one or more disulfide groups is preferably selected from the group consisting of Glutathione Disulfide, Dipyrithione, Selenium Sulfide, Disulfiram, Lipoic Acid, L-Cystine, Fursultiamine, Octreotide, Desmopressin, Vapreotide, Terlipressin, Linaclotide, Peginesatide.

In some embodiments, the analyte molecule comprises one or more epoxide group as functional group. The analyte molecule comprising one or more epoxide groups is preferably selected from the group consisting of Carbamazepine 10,11 epoxide, Carfilzomib, Furosemide epoxide, and Fosfomycin, Sevelamer, Cerulenin, Scopolamine, Tiotropium, Methylscopolamine bromide, Eplerenone, Mupirocin, Natamycin, Carfilzomib, Troleandomycin.

In some embodiments, the analyte molecule comprises one or more phenol groups as functional group. Analyte molecules comprising one or more phenol groups are preferably steroids or steroid-like compounds. In some embodiments, the analyte molecule comprising one or more phenol groups is preferably a steroid or a steroid-like compound having an A-ring which is sp² hybridized and an OH group at the 3-position of the A-ring. The steroid or steroid-like analyte molecule is preferably selected from the group consisting of estrogen, estrogen-like compounds, estrone (E1), estradiol (E2), 17a-estradiol, 17p-estradiol, estriol (E3), 16-epiestriol, 17-epiestriol, and 16,17-epiestriol, and/or metabolites thereof. In embodiments, the metabolites is selected from the group consisiting of estriol, 16-epiestriol (16-epiE3), 17-epiestriol (17-epiE3), 16,17-epiestriol (16,17-epiE3), 16-ketoestradiol (16-ketoE2), 16a-hydroxyestrone (16a-OHE1), 2-methoxyestrone (2-MeOE1), 4-methoxyestrone (4-MeOE1), 2-hydroxyestrone-3-methyl ether (3-MeOE1), 2-methoxyestradiol (2-MeOE2), 4-methoxyestradiol (4-MeOE2), 2-hydroxyestrone (20HE1), 4-hydroxyestrone (4-OHE1), 2-hydroxyestradiol (2-OHE2), estrone (E1), estrone sulfate (E1s), 17a-estradiol (E2a), 17p-estradiol (E2b), estradiol sulfate (E2s), equilin (EQ), 17a-dihydroequilin (EQa), 17p-dihydroequilin (EQb), Eqilenin (EN), 17-dihydroequilenin (ENa) 17b-dihydroequilenin (ENb), A8,9-dehydroestrone (dE1), A8,9-dehydroestrone sulfate (dE1s), D9-Tetrahydrocannabinol, Mycophenolic acid.

In some embodiments, the analyte molecule comprises an amine group as functional group. The amine group is preferably an alkyl-amine or an aryl-amine group. In some embodiments, the analyte comprising one or more amine groups is selected from the group consisting of proteins and peptides. The analyte molecule comprising an amine group is preferably selected from the group consisting of 3,4-Methylendioxyamphetamin, 3,4-Methylendioxy-N-ethylamphetamin, 3,4-Methylenedioxymethamphetamine, Amphetamin, Methamphetamin, N-methyl-1,3-benzodioxolylbutanamine, 7-Aminoclonazepam, 7-amino flunitrazepam, 3,4-Dimethylmethcathinone, 3-Fluoromethcathinone, 4-Methoxymethcathinone, 4-Methylethcathinone, 4-Methylmethcathinone, Amfepramone, Butylone, Ethcathinone, Flephedrone, Methcathinone, Methylone, Methylendioxypyrovaleron, Benzoylecgonine, Dehydronorketamine, Ketamine, Norketamine, Methadone, Normethadone, 6-Acetylmorphine, Diacetylmorphine, Morphine, Norhydrocodone, Oxycodone, Oxymorphone, Phencyclidine, Norpropoxyphene, Amitriptyline, Clomipramine, Dothiepin, Doxepin, Imipramine, Nortriptyline, Trimipramine, Fentanyl, Glycylxylidide, Lidocaine, Monoethylglycylxylidide, N-Acetyl Procainamide, Procainamide, Pregabalin, 2-Methylamino-1-(3,4-methylendioxyphenyl)butan, 2-Amino-1-(3,4-methylendioxyphenyl)butan, Normeperidine, O-Destramadol, Tramadol, Lidocaine, N-Acetyl Procainamide, Procainamide, Gabapentin, Lamotrigine, Theophyllin, Amikacin, Gentamicin, Tobramycin, Vancomycin, Methotrexat, Gabapentin, Sisomicin, and 5-Methylcytosine.

In some embodiments, the analyte molecule is a carbohydrate or substance having a carbohydrate moiety, e.g. a glycoprotein or a nucleoside. The analyte molecule is then preferably a monosaccharide, in particular selected from the group consisting of ribose, desoxyribose, arabinose, ribulose, glucose, mannose, galactose, fucose, fructose, N-acetylglucosamine, N-acetylgalactosamine, neuraminic acid, N-acetylneurominic acid, etc. In some embodiments, the analyte molecule is an oligosaccharide, in particular selected from the group consisting of a disaccharide, trisaccharid, tetrasaccharide, polysaccharide. In some embodiments, the disaccharide is preferably selected from the group consisting of sucrose, maltose and lactose. In some embodiments the analyte molecule is a substance comprising above described mono-, di-, tri-, tetra-, oligo- or polysaccharide moiety.

In some embodiments, the analyte molecule comprises an azide group as functional group which is selected from the group consisting of alkyl or aryl azide. The analyte molecule comprising one or more azide groups is then preferably selected from the group consisting of Zidovudine and Azidocillin.

The functional group Y of the analyte reacts or is able to react with a reactive group X of the derivatisation agent; the reactive groups X of the derivatisation agent are disclosed in detail in the section related to the first aspect of the invention, where also the suitable bonding with the function group Y of the analyte is described. It is understood by a person skilled in the art what the remainders Xa and Ya of X and Y respectively are: in each case, a covalent bond is formed. For example, if the reactive group X of the derivatisation agent is a carbonyl reactive unit such as a hydrazine unit, especially a —NH—NH₂ group, and the functional group of the analyte is a carbonyl group such as a keto group C(═O), a —NH—N═ structure is formed and the remainder Xa of X is —NH—N═ and the remainder Ya of Y is the carbon atom bearing the double bond to the nitrogen atom.

As indicated already above, the chromophore C, which the derivatisation agent carries, is a suitable chromophore enabling an efficient energy transfer during LDI. Furthermore, the derivatisation agent adds a sufficient large molecular weight onto an analyte of interest, wherein the weight addition results in a sufficient mass shift beyond the high noise background of biological samples in the lower molecular weight region. Generally, it is known that such high molecular weight resulted in unfavorable ionization properties (poor ionization efficiency, multiple fragmentation processes . . . ), which is in the present case however circumvented by the presence of a permanent positive charge (Z unit in formula (I)). Therefore, the precursor ion (mother ion) can be detected with high sensitivity and, optionally, be selected for fragmentation in MS/MS applications.

In some preferred embodiments of the conjugate, the conjugate has a molecular weight in the range of from ≥500 g/mol, preferably a molecular weight ≥700 g/mol and/or a M⁺ peak in a mass spectrum of m/z≥500, preferably ≥700. The molecular weight of the conjugate is preferably so high that the resulting M⁺ peak in a mass spectrum lies outside of the range of the low molecular weight background, i.e. the molecular weight of the derivatisation agent, which is coupled to the analyte of interest, is so high that it brings the M⁺ peak of the conjugate outside of the low molecular weight background range.

In some preferred embodiments of the conjugate, the conjugate comprises a neutral loss unit C-L1-Z, wherein C, L1 and Z are as defined above, which has a molecular weight of ≥300 g/mol and/or a peak in a mass spectrum of m/z≥300, preferably a molecular weight of ≥320 g/mol and/or a peak in a mass spectrum of m/z≥320, more preferably a molecular weight of ≥350 g/mol and/or a peak in a mass spectrum of m/z≥350, more preferably a molecular weight of ≥370 g/mol and/or a peak in a mass spectrum of m/z≥370, more preferably a molecular weight of ≥380 g/mol and/or a peak in a mass spectrum of m/z≥380.

The term “neutral loss unit” refers to a unit, which is able to loose a entity having no charge, i.e. which is able to release a neutral entity. Typically, the neutral entity comprises a single atom or a plurality of atoms. A neutral loss unit may be neutral, positively, or negatively charged. A neutral loss unit is, under conditions of MS, capable of fragmentation, whereby at least one neutral entity is released. After release of the neutral entity, the remainder of the neutral loss unit remains its original charge. Accordingly, in case the neutral loss unit is not charged it remains neutral after the loss of the neutral entity. In case the neutral loss unit is positively charged it remains positive after the loss of the neutral entity. In case the neutral loss unit is negatively charged it remains negative after the loss of the neutral entity. Typically, the release of the neutral entity occurs in a single fragmentation event. The term “fragmentation” refers to the dissociation of a single molecule into two or more separate molecules. As used herein, the term fragmentation refers to a specific fragmentation event, wherein the breaking point in the parent molecule at which the fragmentation event takes place is well defined, and wherein the two or more daughter molecules resulting from the fragmentation event are well characterized. It is well-known to the skilled person how to determine the breaking point of a parent molecule as well as the two or more resulting daughter molecules. The resulting daughter molecules may be stable or may dissociate in subsequent fragmentation events. Fragmentation may occur via collision-induced dissociation (CID), electron-capture dissociation (ECD), electron-transfer dissociation (ETD), negative electron-transfer dissociation (NETD), electron-detachment dissociation (EDD), photodissociation, particularly infrared multiphoton dissociation (IRMPD) and blackbody infrared radiative dissociation (BIRD), surface-induced dissociation (SID), Higher-energy C-trap dissociation (HCD), charge remote fragmentation.

As indicated already above, an efficient fragmentation is ensured by the specific combination of Z unit and N unit, which is in some embodiments a quaternary amine group neighboring a benzylic position, which enables a smooth and selective neutral loss fragmentation process. During MS/MS the conjugate of derivatisation reagent and analyte undergoes fragmentation by releasing a large fragment and the production ion (analyte of interest modified with a benzylic cation). This large mass shift is again highly advantageous, because it ensures a low background/interferences of this novel reagent class, since such specific and sensitive neutral loss pathways via large molecular weight loss are very uncommon.

### 5^{th} Aspect—Method for the Mass Spectrometric Determination of an Analyte Molecule

In a fifth aspect, the invention is related to a method for the mass spectrometric determination of an analyte molecule comprising the steps:
(a) providing an analyte of interest;
(b) providing a derivatisation agent comprising a structure of formula (I) as defined in the section related to the first aspect;
(c) reacting the analyte provided according to (a) with the derivatisation agent provided according to (b), whereby a, preferably covalently bound, conjugate of the analyte and the derivatisation agent is formed, and
(d) subjecting the conjugate formed in (c) to a mass spectrometric analysis.

Preferably, the mass spectrometric analysis is laser desorption ionization mass spectrometry (LDI-MS), more preferably is (MA)LDI-MS, more preferably (MA)LDI-MS/MS, or (SA)LDI-MS, more preferably (SA)LDI-MS/MS.

All details, embodiments and preferred embodiments described in the sections above for the first, the second, the third and the fourth aspect of the invention, especially the details, embodiments and preferred embodiments described in the section related to the first aspect of the invention, apply also for the fifth aspect of the invention.

The present invention is further illustrated by the following embodiments and combinations of embodiments as indicated by the respective dependencies and back-references. In particular, it is noted that in each instance where a range of embodiments is mentioned, for example in the context of a term such as “The . . . of any of embodiments 1 to 4”, every embodiment in this range is meant to be explicitly disclosed for the skilled person, i.e. the wording of this term is to be understood by the skilled person as being synonymous to “The . . . of any of embodiments 1, 2, 3, and 4”.
1. Derivatisation agent, preferably derivatisation agent for analytes intended to be analysed via laser desorption ionization mass spectrometry (LDI-MS), comprising a structural element of formula (I)

C-L1-Z-(L2)ₚ-X (I)
wherein
C is a chromophore having an absorption maximum in the range of from 280 to 400 nm;
Z is a charged unit comprising at least one permanently charged moiety;
X is a reactive group;
L1, L2 are each a linker unit; and
p is either zero or 1.
2. The derivatisation agent of embodiment 1, wherein absorption maximum of the chromophore C is an adsorption maximum determined by UV/VIS spectroscopy.
3. The derivatisation agent of embodiment 1 or 2, wherein the absorption maximum of the chromophore C is in the range of from 290 to 380 nm, preferably in the range of from 300 to 360 nm, more preferably in the range of from 305 to 330 nm.
4. The derivatisation agent of any one of embodiments 1 to 3, wherein the chromophore C has a structure of formula (C)

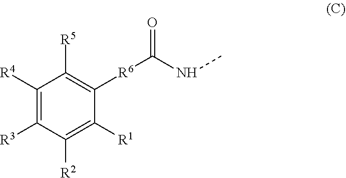

wherein R¹, R², R³, R⁴, R⁵ are independently selected from the group of hydrogen atom; hydroxyl group; NR^{x}R^{y} group, wherein R^{x} and R^{y} are independently a hydrogen atom or a C1 to C5 alkyl group; C1 to C5 alkyl, C5 to C10 (hetero)aryl and —O—C1 to C3 alkoxy group; and R⁶ is either none (i.e. there is a direct covalent single bond between the aromatic cycle and the C(═O) group) or is a —CR⁷═CR⁸— group, wherein R⁷ is a hydrogen atom or a C1 to C3 alkyl group and R⁸ is selected from the group consisting of hydrogen atom, C1 to C5 alkyl group and electron withdrawing group; and the dotted line represent a bond to the linker, which is preferably a single bond.
5. The derivatisation agent of any one of embodiments 1 to 4, wherein the chromophore C has a structure of formula (C1), (C2) or (C3):

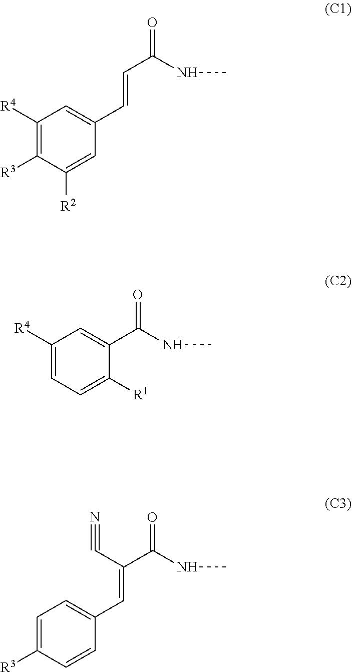

wherein R¹, R², R³, R⁴, are independently selected from the group of hydrogen atom, hydroxyl group NR^{x}R^{y} group, wherein R^{x} and R^{y} are independently a hydrogen atom or a C1 to C5 alkyl group, C1 to C5 alkyl, C5 to C10 (hetero)aryl and —O—C1 to C3 alkoxy group.
6. The derivatisation agent of embodiment 5, wherein the chromophore C has a structure of formula (C1), wherein R¹ and R⁴ are both a methoxy group and R³ is a hydroxyl group (C1 with these residues R², R³ and R⁴ being the remainder of sinapinic acid).
7. The derivatisation agent of embodiment 5, wherein the chromophore C has a structure of formula (C2), wherein R² and R⁴ are both a hydroxyl group (C2 with these residues R², R⁴ being the remainder of 2,5-dihydroxy benzoic acid).
8. The derivatisation agent of embodiment 5, wherein the chromophore C has a structure of formula (C3), wherein R³ is a hydroxyl group (C3 with this residue R³ being the remainder of alpha-cyano-4-hydroxy-cinamix acid).
9. The derivatisation agent of any one of embodiments 1 to 8, wherein the linker L1 is selected from the group consisting of (C1-C5 alkylene-O—)ₘ group with m being an integer in the range of from 1 to 10, C1 to C20 alkylene group, C1 to C20 alkylene group-heteroaryl group and (C1-C5 alkylene)-O—(C1-C5 alkylene) group, optionally connected to or intersected by a unit selected from the group consisting of (hetero)aryl group, N₂, NO, NO₂, S₂, SO, SO₂, CO, and CO₂, said unit being preferably, if present, a heteroaryl group, more preferably from triazol, phenyltriazol, tetrazol and phenyltetrazol.
10. The derivatisation agent of any one of embodiments 1 to 9, wherein the linker L1 has a structure (L1a)_{q}-(L1b)ᵣ-(L1c)ₛ, wherein q, r, s are each zero or 1, with the condition that at least one of q, r, s is 1; L1a being selected from the group consisting of (C1-C5 alkylene-O—)ₘ group with m being an integer in the range of from 1 to 10, C1 to C10 alkylene group; L1b being a unit selected from the group consisting of N₂, NO, NO₂, S₂, SO, SO₂, CO, CO₂, triazol, phenyltriazol, tetrazol and phenyltetrazol, wherein the unit is preferably a triazol or a tetrazol, more preferably the neutral loss unit N is a triazol, more preferably the neutral loss unit N is a 1,2,3 triazol ring, which is bound to the linker L1a, if present, via a single bond at position 1 (N atom) of the triazol ring and bound to the Linker L1c, if present, via a single bond at position 4 (C atom) of the triazol ring; L1c, if present, being selected from the group consisting of (C1-C5 alkylene-O—)ₘ group with m being an integer in the range of from 1 to 10, C1 to C10 alkylene group and (C1-C5 alkylene)-O—(C1-C5 alkylene) group.
11. The derivatisation agent of any one of embodiments 1 to 10, wherein the linker L1 has a structure

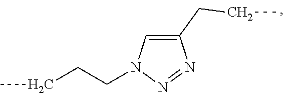

wherein the dotted lines indicate the bonds to (C) and (Z) respectively.
12. The derivatisation agent of any one of embodiments 1 to 11, wherein the charged unit Z is positively or negatively charged, preferably positively charged.
13. The derivatisation agent of any one of embodiments 1 to 12, wherein the charged unit Z is positively charged and is preferably a tetraalkyl ammonium group, more preferably a —CH₂N⁺(CH₃)₂CH₂— group.
14. The derivatisation agent of any one of embodiments 1 to 12, wherein the charged unit Z is negatively charged, wherein the negatively charged unit Z is preferably selected from the group consisting of a phosphate, sulphate, sulphonate and carboxylate.
15. The derivatisation agent of any one of embodiments 1 to 12, wherein the linker L2 comprises 1 to 10 C atoms and optionally one or more heteroatom(s).
16. The derivatisation agent of embodiment 15, wherein the linker L2 is a C1-C5-alkylene-C5 to C10 aromatic ring, which preferably bears a C(═O) unit as substituent of the aromatic ring, the linker L2 preferably being a C1-C3-alkylene-C6 aromatic ring, which preferably bears a C(═O) unit as substituent of the aromatic ring, wherein the C6 aromatic ring is more preferably a substituted or unsubstituted benzene ring, more preferably benzene ring, which bears a C(═O) group at the position of the benzene ring which is para with respect to the position where the C1-C3-alkylene is bound and which more preferably bears no further substituents.
17. The derivatisation agent of any one of embodiments 1 to 16, wherein the reactive group X is selected from the group consisting of carbonyl reactive unit, diene reactive unit, hydroxyl reactive unit, amino reactive unit, imine reactive unit, thiol reactive unit, diol reactive unit, phenol reactive unit, epoxide reactive unit, disulfide reactive unit, and azido reactive unit.
18. A kit comprising the derivatisation agent according to any one of embodiments 1 to 17.
19. Use of a derivatisation agent according to any one of embodiments 1 to 17 for the mass spectrometric determination of an analyte molecule, wherein the mass spectrometric determination is laser desorption ionization mass spectrometry (LDI-MS), preferably is (MA)LDI-MS, more preferably (MA)LDI-MS/MS, or(SA)LDI-MS, more preferably (SA)LDI-MS/MS.
20. A conjugate of a derivatisation agent according to any one of embodiments 1 to 17 and an analyte, wherein the conjugate has the structure of formula (II)

C-L1-Z-(L2)ₚ-Xa-Ya-A (II)
wherein C, L1, L2, p, Z and N are as defined in any one of embodiments 1 to 17; Xa is a remainder of a reactive group X as defined in any one of embodiments 1 to 17; A is the analyte and Ya is the remainder of a reactive group Y bound to the analyte A, which has reacted with the reactive group X of the derivatisation agent thus forming a covalent bound between Xa and Ya.
21. The conjugate of embodiment 20, wherein the analyte.is selected from the group consisting of nucleic acid (preferably selected from DNA, mRNA, miRNA, and rRNA), amino acid, peptide, protein (preferably cell surface receptor or cytosolic protein), metabolite, hormone (preferably selected from testosterone, estrogen and estradiol), fatty acid, lipid, carbohydrate, steroid, ketosteroid, secosteroid (Vitamin D), molecule characteristic of a certain modification of another molecule (preferably selected from sugar moiety, phosphoryl residue on a protein, methyl-residue on genomic DNA), substance that has been internalized by the organism (preferably selected from therapeutic drug, drug of abuse, toxin) and a metabolite of such a substance.
22. The conjugate of embodiment 20 or 21, wherein the reactive group Y . . . is selected from the group consisting of carbonyl group, diene group, hydroxyl group, amine group, imine group, thiol group, diol group, phenolic group, expoxid group, disulfide group, and azide group.
23. The conjugate of any one of embodiments 20 to 22 having a molecular weight in the range of from ≥500 g/mol, preferably a molecular weight ≥700 g/mol and/or a M⁺ peak in a mass spectrum of m/z≥500, preferably ≥700.
24. The conjugate of any one of embodiments 20 to 22 comprising a neutral loss unit C-L1-Z, which has a molecular weight of ≥300 g/mol and/or a peak in a mass spectrum of m/z≥300, preferably a molecular weight of ≥320 g/mol and/or a peak in a mass spectrum of m/z≥320, more preferably a molecular weight of ≥350 g/mol and/or a peak in a mass spectrum of m/z≥350, more preferably a molecular weight of ≥370 g/mol and/or a peak in a mass spectrum of m/z≥370, more preferably a molecular weight of ≥380 g/mol and/or a peak in a mass spectrum of m/z≥380.
25. A method for the mass spectrometric determination of an analyte molecule comprising the steps:
   (e) providing an analyte of interest;
   (f) providing a derivatisation agent comprising a structure of formula (I) as defined in any one of embodiments 1 to 17;
   (g) reacting the analyte provided according to (a) with the derivatisation agent provided according to (b), whereby a, preferably covalently bound, conjugate of the analyte and the derivatisation agent is formed, and
   (h) subjecting the conjugate formed in (c) to a mass spectrometric analysis.
26. The method for the mass spectrometric determination, wherein the mass spectrometric analysis is laser desorption ionization mass spectrometry (LDI-MS), preferably is (MA)LDI-MS, more preferably (MA)LDI-MS/MS, or (SA)LDI-MS, more preferably (SA)LDI-MS/MS.

The present invention is further illustrated by the following reference examples, comparative examples, and examples.

### EXAMPLES

### (Comparative) Example 1: Synthesis of [4-(hydrazinecarbonyl)phenyl]-N,N,N-trimethyl-methanaminium bromide (alkyne 3)

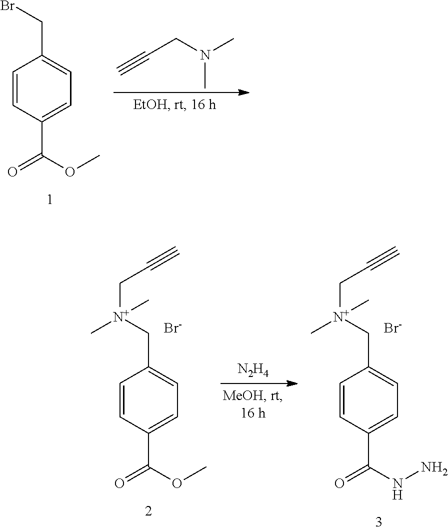

Bromide 1 (2.00 g, 8.73 mmol) and N,N-dimethylpropargylamine (4.8 ml, 64.1 mmol) were dissolved in 30 ml EtOH and the reaction mixture was stirred at room temperature for 16 h. The precipitate was collected by filtration, washed with EtOH and Et₂O and dried in vacuo. The desired product 2 was obtained as a yellow solid (2.73 g, quant. yield).

¹H-NMR (400 MHz, METHANOL-d4): δ[ppm]=3.19 (s, 3H), 3.68 (t, 1H), 3.93 (s, 3H), 4.31 (d, 2H), 4.74 (s, 2H), 7.75 (d, 2H), 8.16 (d, 2H).

¹³C-NMR (101 MHz, METHANOL-d4): δ[ppm]=49.47, 51.56, 53.43, 65.89, 71.10, 82.48, 129.87, 131.72, 132.37, 132.90, 166.08.

ESI-MS: 232.2 ([M⁺]⁺, calc.: 232.3)

Hydrazine monohydrate (6.8 ml, 64.1 mmol) was added to a solution of tertiary amide 2 (2.00 g 6.41 mmol) in 30 ml MeOH and the reaction mixture was stirred at room temperature. After 16 h, the reaction mixture was concentrated in vacuo and the crude product was purified by preparative RP-HPLC using an isocratic mobile phase (100% water). The desired product 3 was obtained as a yellow oil (1.69 g, 5.41 mmol, 84%).

¹H-NMR: (400 MHz, METHANOL-d4): δ[ppm]=3.19 (s, 3H), 3.68 (t, 1H), 4.28 (d, 2H), 4.71 (s, 2H), 7.58 (d, 2H), 7.86 (d, 2H).

¹³C-NMR (101 MHz, METHANOL-d4): δ[ppm]=42.13, 49.41, 53.32, 60.70, 65.97, 71.13, 82.44, 127.72, 132.86, 133.96, 134.78, 168.21.

ESI-MS: 232.2 ([M+]+, calc.: 232.3).

### Example 1: Synthesis of Derivatisation Agent 5a

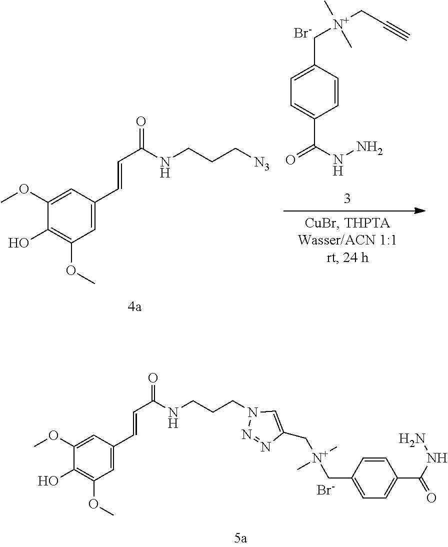 [SMILES: COc1cc(/C=C/C(=O)NCCCN=[N+]=[N-])cc(OC)c1O ; C#CC[N+](C)(C)Cc1ccc(C(=O)NN)cc1 ; [Br-] ; COc1cc(/C=C/C(=O)NCCCn2cc(C[N+](C)(C)Cc3ccc(C(=O)NN)cc3)nn2)cc(OC)c1O ; [Br-]]

To a solution of CuBr (16 mg, 0.10 mmol) and THPTA (44 mg, 0.10 mmol) in THF/water (2 ml, 1/1 (v/v)) was added a solution of alkyne 3 (310 mg, 1.0 mmol) and azide 4 (101 mg, 0.33 mmol) in THF/water (2 ml, 1/1 (v/v)) under argon atmosphere. The reaction mixture was stirred at room temperature for 24 h and subsequently concentrated in vacuo. The crude product was purified by preparative RP-HPLC using a linear gradient from water/acetonitrile 100/0->0/100 in 60 min. The desired product 5a was obtained as a beige solid (90 mg, 0.15 mmol, 44%) after lyophilization.

¹H-NMR: (400 MHz, DMSO-d6): δ[ppm]=2.05 (m, 3H), 2.91 (m, 5H), 3.18 (m, 3H), 3.76 (m, 5H), 4.57 (m, 5H), 6.51 (d, 1H), 6.82 (s, 1H), 7.28 (d, 1H), 7.75 (d, 2H), 7.94 (d, 2H), 8,27 (m, 1H), 8.53 (m, 1H)

ESI-MS: 538.5 ([M+]+, calc.: 538.6).

### Example 2: Coupling of Derivatisation Agent 5a With Analyte (Testosterone)—Synthesis of Conjugate 6a

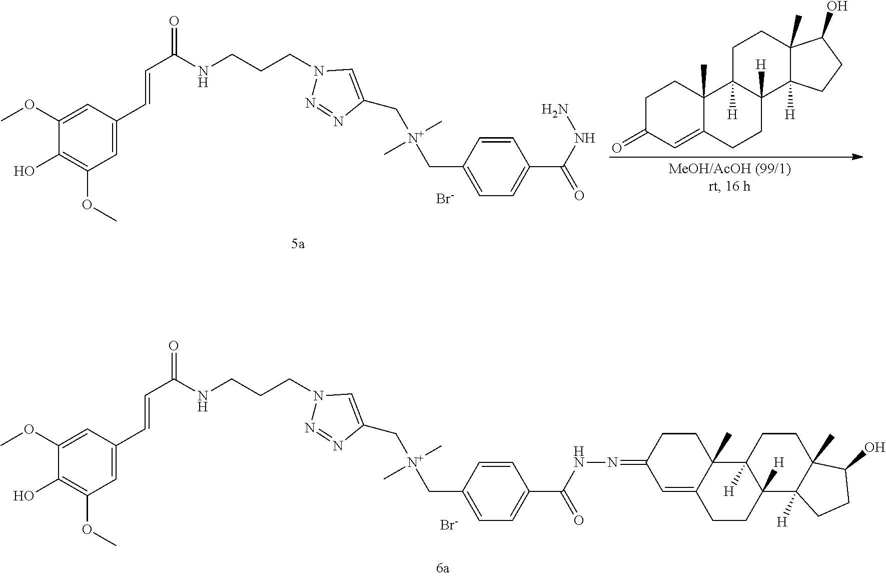

Hydrazide 5a (55 mg, 90.0 μmol) and testosterone (77 mg, 270 μmol) were dissolved in 1 ml methanol/formic acid (99/1, v/v). The reaction mixture was stirred at room temperature for 16 h and subsequently concentrated in vacuo. The crude product was purified by preparative RP-HPLC using a linear gradient from water/acetonitrile 100/0->0/100 in 60 min. The desired hydrazone 6a was obtained as a beige solid (35.6 mg, 40.0 μmol, 44%) after lyophilization.

¹H-NMR: (400 MHz, METHANOL-d4): δ[ppm]=0.50-2.45 (m, 27H), 2.91 (m, 5H), 3.17 (m, 3H), 3.76 (s, 4H), 4.47 (m, 2H), 4.59 (m, 2H), 4.66 (m, 1H), 5.60 (s, 1H), 6.49 (d, 1H), 6.81 (s, 2H), 7.28 (d, 1H), 7.72 (d, 2H), 7.94 (d, 2H), 8.28 (s, 1H), 8.50 (m, 1H).

ESI-MS: 808.5 ([M+]+, calc.: 808.5).

### Example 3: Synthesis of Derivatisation Agent 5b

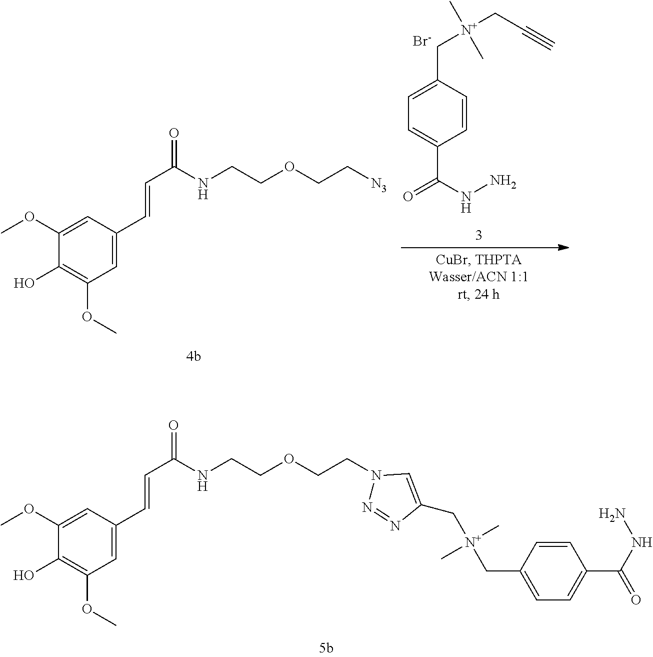 [SMILES: COc1cc(/C=C/C(=O)NCCOCCN=[N+]=[N-])cc(OC)c1O ; C#CC[N+](C)(C)Cc1ccc(C(=O)NN)cc1 ; [Br-] ; COc1cc(/C=C/C(=O)NCCOCCn2cc(C[N+](C)(C)Cc3ccc(C(=O)NN)cc3)nn2)cc(OC)c1O]

To a solution of CuBr (16 mg, 0.10 mmol) and THPTA (44 mg, 0.10 mmol) in THF/water (2 ml, 1/1 (v/v)) was added a solution of alkyne 3 (310 mg, 1.0 mmol) and azide 4b (111 mg, 0.33 mmol) in THF/water (2 ml, 1/1 (v/v)) under argon atmosphere. The reaction mixture was stirred at room temperature for 24 h and subsequently concentrated in vacuo. The crude product was purified by preparative RP-HPLC using a linear gradient from water/acetonitrile 100/0->0/100 in 60 min. The desired product 5b was obtained as a beige solid (138 mg, 0.21 mmol, 64%) after lyophilization.

ESI-MS: 568.4 ([M+]+, calc.: 568.6).

### Example 4: Coupling of Derivatisation Agent 5b With Analyte (Testosterone)—Synthesis of Conjugate 6b

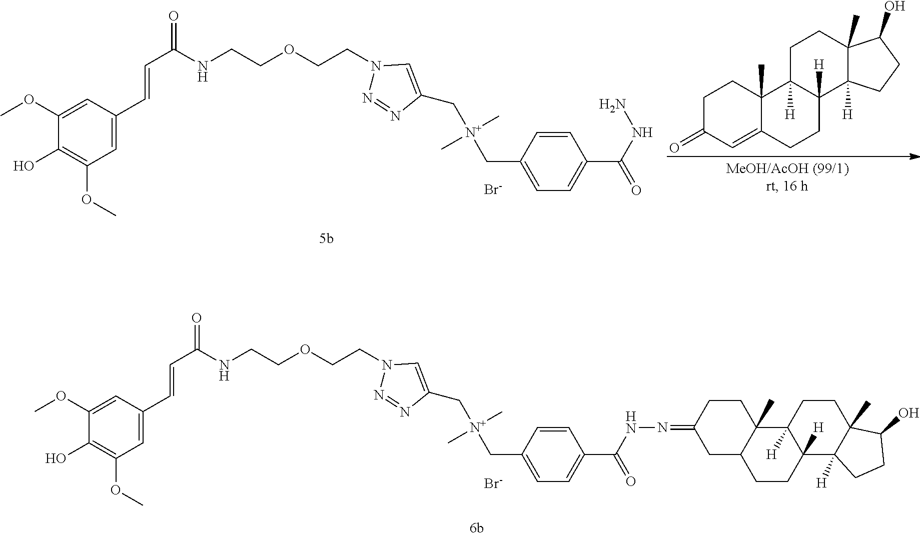

Hydrazide 5b (115 mg, 0.18 mmol) and testosterone (153 mg, 0.53 mmol) were dissolved in 1 ml methanol/formic acid (99/1, v/v). The reaction mixture was stirred at room temperature for 16 h and subsequently concentrated in vacuo. The crude product was purified by preparative RP-HPLC using a linear gradient from water/acetonitrile 100/0->0/100 in 60 min. The desired hydrazone 6b was obtained as a beige solid (54.0 mg, 58.8 μmol, 32%) after lyophilization.

¹H-NMR: (400 MHz, METHANOL-d4): δ[ppm]=0.56-2.40 (m, 20H), 2.89 (s, 3H), 3.76 (s, 3H), 3.83 (m, 1H), 4.60 (m, 3H), 6.52 (m, 1H), 6.83 (s, 1H), 7.28 (d, 1H), 7.70 (m, 2H), 7.95 (m, 2H), 8.12 (s, 1H), 8.47 (m, 2H).

ESI-MS: 838.5 ([M+]+, calc.: 838.5).

### Comparative Example 2: Coupling of Derivatisation Agent 3 With Analyte (Testosterone)—Synthesis of Conjugate 6c

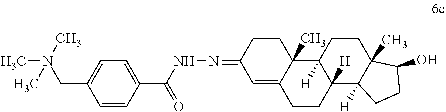

Alkyne 3 and testosterone (153 mg, 0.53 mmol) were reacted to give conjugate 6c.

### Example 5: Laser-Desorption/Ionization Mass Spectrometric Analysis

### 5a Sample Preparation and General Measurement Details

Stock solutions of all relevant analytes 6a, 6b, 6c and testosterone were prepared individually in concentrations of 1.0 mg/ml (100% acetonitrile; ACN) and further diluted to reach individual molar concentrated solutions of 12 μM (80/10=ACN/H₂O; abbreviated as 80% ACN). An analyte mix was prepared from the molar concentrated stock solutions, yielding a molar concentration of 3 μM (80% ACN) of each analyte. Further dilutions of this analyte mix solution in 300 nM, 30 nM and 3.0 nM (80% ACN) were prepared.

For sinapinic acid matrix experiments, a matrix solution was freshly prepared, consisting of 10 mg/ml sinapinic acid in 50% ACN, 0.1% formic acid (FA).

As MALDI target plate, a common 96-well steel plate (Waters Corp.) was used. Similar to this, the SALDI-MS target plate is also based on a steel plate, but with a functional amorphous a-C:H:Si:X (X=heteroatom modified) plasma activated chemical vapor deposited (PACVD) surface coating as top layer.

All following laser-desorption/ionization mass spectrometric (LDI-MS) measurements have been performed on a MALDI Synapt G2-Si (Waters Corp.) MALDI-Q-ToF mass spectrometer in positive ion mode. The laser repetition rate of the LDI-MS system was set to 1.0 kHz for the MALDI experiments and to 200 Hz for the SALDI experiments without matrix, utilizing a Nd:YAG-laser wavelength of 355 nm. Analyte spots were each measured by a random path over the whole analyte spot area, utilizing a plate movement of 12 Hz during the laser irradiation. The total acquisition time for each measurement was set to 30 s with a 0.5 s mass spectral scan time. The laser intensity can be varied in a relative scale of up to 500—resembling a maximum output energy of 30 μJ—while the optimal laser energy of the sinapinic acid MALDI experiments was found to be 280 units and the optimal laser energy of the matrix free SALDI experiments was increased to 380 units. Individual voltage settings are outlined in detail with the respective measurements.

The corresponding data analysis was performed using MassLynx 4.2 (SCN983, Waters Corp.) mass spectrometric instrument software. All obtained mass spectral functions of each sample spot were accumulated over the whole 30 s analysis time.

Acquisition parameters of MALDI-measurements:

| Polarity | LDI+ | |
|---|---|---|
| Start mass | m/z 200 | |
| End mass | m/z 1000 | |
| Acquisition time | 30 | s |
| Cycle time | 0.514 | s |
| Scan time | 0.500 | s |
| Inter Scan Delay | 0.014 | s |
| Data Format | Continuum | |
| Analyser | Sensitivity Mode | |
| Maldi Plate Speed | 12.0 | Hz |
| Maldi Laser Firing Rate | 1000 | Hz |
| Maldi Laser Energy | 280.0 | units |
| Sample Plate | 0.0 | V |
| Maldi Extraction | 10.0 | V |
| Hexapole Bias | 10.0 | V |
| Aperture 0 | 5.0 | V |
| hexapole RF amplitude | 350 | V |

| | |
|---|---|
| LM Resolution | 4.4 |
| HM Resolution | 15.0 |

Acquisition parameters of the respective SALDI-measurement:

| Polarity | LDI+ | |
|---|---|---|
| Start mass | m/z 50 | |
| End mass | m/z 1000 | |
| Acquisition time | 30 | s |
| Cycle time | 0.514 | s |
| Scan time | 0.500 | s |
| Inter Scan Delay | 0.014 | s |
| Data Format | Centroid | |
| Analyser | Sensitivity Mode | |
| Maldi Plate Speed | 12.0 | Hz |
| Maldi Laser Firing Rate | 200.0 | Hz |
| Maldi Laser Energy | 380.0 | units |
| Sample Plate | 0.0 | V |
| Maldi Extraction | 10.0 | V |
| Hexapole Bias | 10.0 | V |
| Aperture 0 | 5.0 | V |

| | |
|---|---|
| Cooling Gas Flow | 50.0 |
| LM Resolution | 4.4 |
| HM Resolution | 15.0 |

### 5b: Evaluation of Derivatisation Reagents in Matrix Assisted Laser-Desorption/Ionization Mass Spectrometric Analysis (MALDI-MS)

At first the suitability of isolated derivatized testosterone conjugates 6a and 6b was tested for a possible matrix assisted laser-desorption/ionization mass spectrometric (MALDI-MS) application. The performance of the two chromophore-containing conjugates 6a and 6b was compared against native testosterone (T) and the derivatized testosterone conjugate 6c, which serves as a gold standard (in the field of liquid chromatography based MS-applications) but does not contain a LDI suitable chromophore. Equimolar four-analyte-mixes (containing all four analytes 6a, 6b, 6c and testosterone (T)) were prepared with different concentrations (3 μM, 300 nM, 30 nM and 3 nM) and subsequently premixed (1:1) with a sinapinic acid matrix solution. Next, 1 μl of this analyte-matrix solution was applied to a MALDI target steelplate by dried-droplet preparation. The obtained analyte-matrix crystals were measured by MALDI-MS in full scan positive ion mode (m/z 200 to m/z 1000; further details see experimental section). As expected, analysis of the full scan MALDI mass spectras (see FIG. 1) indicated only a very low signal intensity for the m/z corresponding to the native analyte ([TH]⁺, protonated testosterone cation). In comparison to that, the derivatized testosterone conjugates 6a and 6b were detected with significantly higher intensity (˜96-fold signal enhancement, see FIG. 2). In comparison to the state-of-the-art conjugate 6c it is clearly visible, that the novel reagents generate detectable ions with a m/z, which are out of the low molecular weight matrix-background (in this experiment m/z=200-700) and therefore are less vulnerable to matrix-based interference. This solves a common problem of MALDI-MS (Anal Bioanal Chem 410, 4015-4038 (2018)).

FIG. 2 shows dilution series of an equimolar (3 μM, 300 nM, 30 nM and 3 nM) analyte mix consisting of derivatized compounds [6a]⁺, [6b]⁺ and [6c]⁺ in comparison to underivatized Testosterone [TH]⁺ in the presence of sinapinic acid MALDI-matrix. The number of detected counts are displayed in a logarithmic scale against the corresponding molar concentration.

However, application of chromophore-carrying conjugates 6a and 6b in sinapinic acid did not result in a signal enhancement in comparison to the state-of-the-art conjugate 6c at concentrations lower than 300 nM. This observation indicated, that in presence of a suitable matrix, in this case sinapinic acid, the energy transfer of the laser beam to the analyte of interest was mediated via the matrix and not via the chromophore-equipped derivatisation reagent, which was conclusive considering the large excess of matrix molecules versus the derivatized analytes. In summary, the herein described chromophore-equipped derivatisation reagents provided for MALDI-MS applications primary a “mass-tag” advantage, which allowed the detection of low molecular weight analytes in absence of matrix-induced low-molecular weight interferences.

### 5c: Evaluation of Derivatisation Reagents in Laser-Desorption/Ionization Mass Spectrometric Analysis (LDI-MS) on a Functional Coated SALDI Plate

As intended by the overall conceptual design, the isolated derivatized testosterone conjugates 6a and 6b were evaluated for laser-desorption/ionization mass spectrometric (MALDI-MS) applications. The performance of the two chromophore-containing conjugates 6a and 6b were again compared against native testosterone (T) and the derivatized testosterone conjugate 6c, which does not contain a LDI suitable chromophore. Equimolar four-analyte-mixes (containing all four analytes 6a, 6b, 6c and testosterone (T)) with different concentrations (3000 nM, 300 nM, 30 nM and 3 nM) were prepared and directly applied to a functionalized SALDI-MS target plate by dried-droplet preparation. The obtained spots were measured by SALDI-MS in full scan positive ion mode (m/z 50 to m/z 1000; further details see experimental section). As expected, analysis of the full scan LDI mass spectras (see example at 3 μM concentration in FIG. 3) indicated an increased number of counts for m/z of the derivatized testosterone-conjugates 6a and 6b in comparison to the native analyte ([TH]⁺, protonated testosterone cation), which corresponds to a ˜15-fold signal enhancement at 3 μM concentration.

FIG. 4 shows dilution series of an equimolar (3 μM, 300 nM, 30 nM and 3 nM) analyte mix consisting of derivatized compounds [6a]⁺, [6b]⁺ and [6c]⁺ in comparison to underivatized Testosterone [TH]⁺ in the presence of sinapinic acid MALDI-matrix. The number of detected counts are displayed in a logarithmic scale against the corresponding molar concentration.

This signal enhancement was especially beneficial for high sensitive measurements at low concentrations. As an example, native testosterone was not detectable at 3 nM, but detectable after derivatisation with reagent 5a and 5b (as [6a]⁺ and [6b]⁺) at this low concentration. It is important to note, that using the LDI-compatible derivatisation reagents 5a and 5b helped to increase the signal of the corresponding testosterone conjugates (as [6a]⁺ and [6b]⁺) in comparison to the state-of-the-art conjugate 6c, which lacked a LDI-compatible chromophore. In comparison [6c]⁺ the absolute number of detected counts were 1.4 and 2.1-fold higher for [6a]⁺ and [6b]⁺, respectively. More importantly the S/N was significantly improved for [6a]⁺ (S/N=304) and [6b]⁺ (S/N=39) at 3 nM in comparison [6c]⁺ (S/N=2). This findings supported the fundamental working hypothesis, that using a permanently charged derivatisation reagent, which is carrying a LDI-compatible chromophore, helped to improve the energy transfer of the laser beam to the derivatized-analyte of interest and thereby increased the desorption/ionization efficiency, which finally led to enhanced sensitivity (J Am Soc Mass Spectrom 2007, 18, 9, 1582-1590). Consequently, the herein described LDI-compatible derivatisation reagents 5a and 5b provided a technical advantage for achieving high-sensitive LDI-MS applications.

### Example 6 Generalisation of the Chromophore

The tested derivatisation reagents 5a-5c were characterized by UV-spectroscopy. The sinapinic acid derived reagents 5a and 5b displayed an absorption maximum of 307 nm and 315 nm, respectively, which is still comparable to the routinely used sinapinc acid. Since the absorption maximas of these two compounds is relatively close (<40 nm) to the wavelength of the laser beam (355 nm), efficient energy transfer is to be expected. This clearly indicates that 5a and 5b are suitable for LDI-applications due to their ability to absorb the energy of the laser beam (typically 355 nm (J Mass Spectrom. 2021; 56:4664)), which is regarded as an essential factor for MALDI matrices (Chem. Rev. 2003, 103, 2, 395-426). Reagent 5c has a local absorption maximum if 240 nm, which is more than 100 nm below the routinely used Nd:YAG laser wavelength. Based on our understanding and the available data, this leads to less spectral overlap with the laser beam and consequently a lower desorption/ionization efficiency, compared to 5a and 5b.

It is known that other small molecules (i.e. 2,5 dihydroxy benzoic acid and alpha-cyano-4-hydroxy-cinnamic acid) with a similar local absorption maximum, but with a different chemical structure (vs sinapinic acid) are also suitable for (MA)LDI processes (see Table 1). Following this data, it was concluded that the presented concept is not limited to structures 5a and 5b, but applicable to any other derivatisation reagent equipped with a chromophore C, which is a suitable LDI-chromophore.

TABLE 1
UV/VIS absorption maxima

| | absorption | |
|---|---|---|
| compound | maximum [nm] | Ref |
| sinapinic acid | 323 | own data |
| 2,5 dihydroxy benzoic acid | 327 | own data |
| alpha-cyano-4-hydroxy- | 325 | Molecules 25(24): 6054 |
| cinnamic acid | | |
| 5a | 307 | own data |
| 5b | 315 | own data |
| 5c | 240 | own data |

### SHORT DESCRIPTION OF THE FIGURES

FIG. 1 shows a full scan MALDI mass spectrum (m/z 200 to m/z 1000) of an equimolar (3 μM) analyte mix consisting of derivatized compounds 6a, 6b, 6c and native testosterone in a sinapinic acid MALDI-matrix.

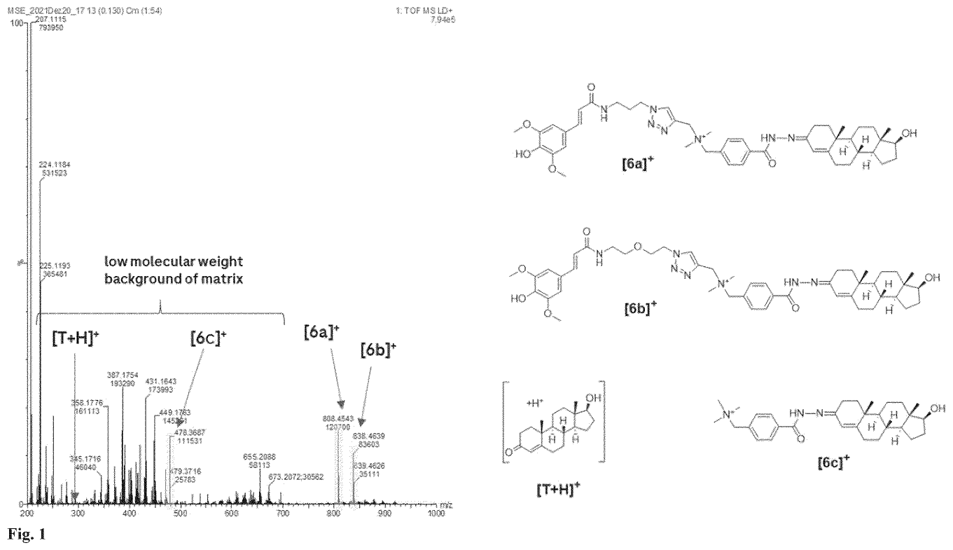

FIG. 2 shows dilution series of an equimolar (3 μM, 300 nM, 30 nM and 3 nM) analyte mix consisting of derivatized compounds [6a]⁺, [6b]⁺ and [6c]⁺ in comparison to underivatized Testosterone [TH]⁺ in the presence of sinapinic acid MALDI-matrix. The number of detected counts are displayed in a logarithmic scale against the corresponding molar concentration.

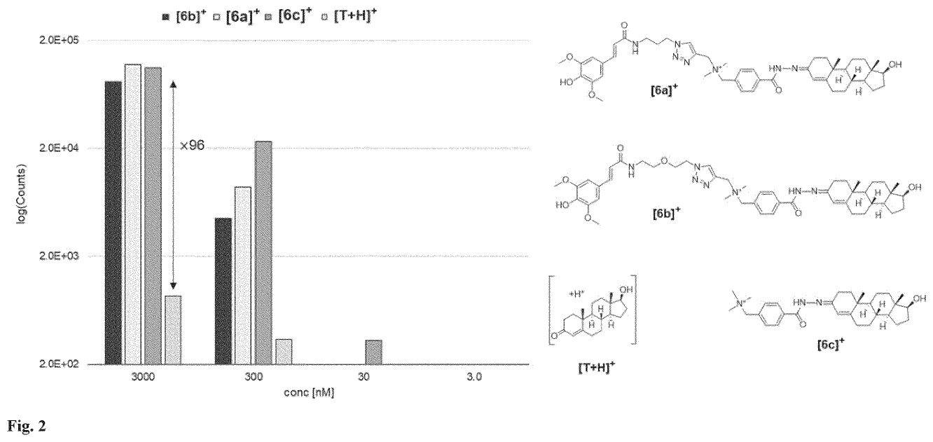

FIG. 3 shows a full scan SALDI mass spectrum (m/z 50 to m/z 1000) of an equimolar (3 μM) analyte mix consisting of derivatized compounds [6a]⁺, [6b]⁺ and [6c]⁺ in comparison to underivatized Testosterone [TH]⁺ without the use of an additional MALDI matrix. *: In-source fragmentation of [6a]⁺, [6b]⁺ and [6c]⁺.

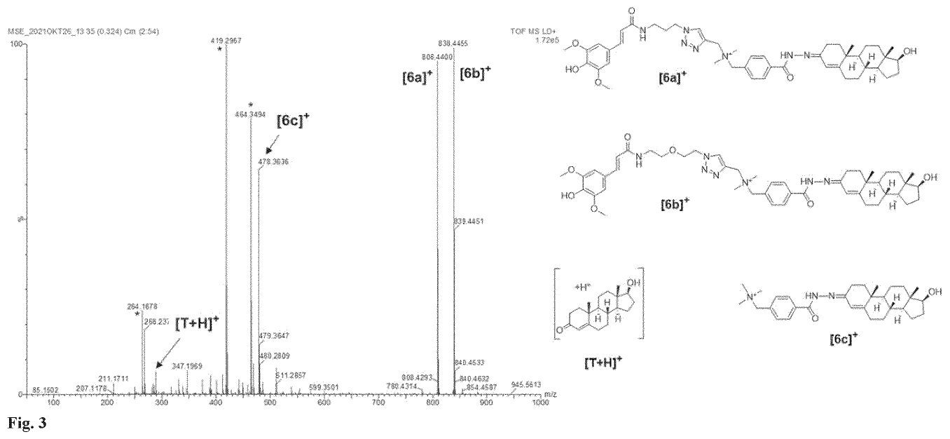

FIG. 4 shows dilution series of an equimolar (3 μM, 300 nM, 30 nM and 3 nM) analyte mix consisting of derivatized compounds [6a]⁺, [6b]⁺ and [6c]⁺ in comparison to underivatized Testosterone [TH]⁺ in the presence of sinapinic acid MALDI-matrix. The number of detected counts are displayed in a logarithmic scale against the corresponding molar concentration.

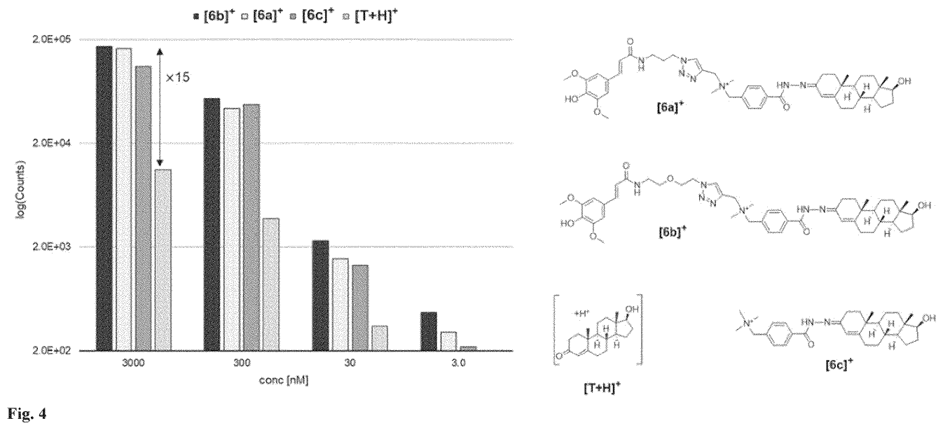

### CITED LITERATURE

Higashi et al. (2016) J. of Pharmaceutical and Biomedical Analysis 130 p. 181-190
WO 2011/091436 A1
WO 2011/091436 A1
Rahimoff et al. (2017) J. Am. Chem. Soc. 139(30), p. 10359-10364
J. Sep. Sci. 2016, 39, 102-114; Biomed. Chromatogr. 2011; 25: 1-10
Trends in Analytical Chemistry 143 (2021) 116399;
J Mass Spectrom. 2021; 56:e4731
J. Sep. Sci. 2016, 39, 102-114
Biomed. Chromatogr. 2011; 25: 1-10
Anal. Chem. 2020, 92, 6224-6228
Chem Asian J. 2021, 16, 868-878
Crit Rev Anal Chem. 2021 Dec. 30; 1-17
Mass Spec Rev. 2019; 38:3-21
Scientific Reports|5:17853, ChemBioChem 2021, 22, 1430-143
International Journal of Mass Spectrometry 353 (2013) 54-59
Anal. Chem. 2014, 86, 21, 10724-10731
T. Higashi J Steroid Biochem Mol Biol. 2016 September; 162:57-69
H. Ban et al J. Am. Chem. Soc., 2010, 132 (5), pp 1523-1525
Anal. Bioanal. Chem. 2011, 399, 2597, DOI 10.1007/s00216-010-4063-3
Anal Bioanal Chem. 2018, 410, 4015-4038
J Am Soc Mass Spectrom 2007, 18, 9, 1582-1590

1. A derivatization agent comprising a structural element of formula (I)
C-L1-Z-(L2)ₚ-X (I)
wherein
C is a chromophore having an absorption maximum in the range of from 280 to 400 nm;
Z is a charged unit comprising at least one permanently charged moiety;
X is a reactive group;
L1, L2 are each a linker unit; and
p is either zero or 1.

2. The derivatization agent of claim 1, wherein the absorption maximum of the chromophore C is an adsorption maximum determined by UV/VIS spectroscopy.

3. The derivatization agent of claim 1, wherein the chromophore C has a structure of formula (C) 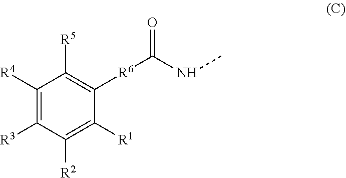
wherein R¹, R², R³, R⁴, R⁵ are independently selected from the group consisting of a hydrogen atom, a hydroxyl group, an NR^{x}R^{y} group, a C1 to C5 alkyl, a C5 to C10 (hetero)aryl, and an —O—C1 to C3 alkoxy group; wherein R^{x} and R^{y} are independently a hydrogen atom or a C1 to C5 alkyl group; wherein R⁶ is either none or is a —CR⁷═CR⁸— group; wherein R⁷ is a hydrogen atom or a C1 to C3 alkyl group; wherein R⁸ is selected from the group consisting of a hydrogen atom, a C1 to C5 alkyl group, and an electron withdrawing group; and wherein the dotted line represents a bond to the linker.

4. The derivatization agent of claim 1, wherein the linker L1 is selected from the group consisting of a (C1-C5 alkylene-O—)ₘ group with m being an integer in the range of from 1 to 10, a C1 to C20 alkylene group, a C1 to C20 alkylene group-heteroaryl group, and a (C1-C5 alkylene)-O—(C1-C5 alkylene) group, optionally connected to or intersected by a unit selected from the group consisting of a (hetero)aryl group, N₂, NO, NO₂, S₂, SO, SO₂, CO, and CO₂.

5. The derivatization agent of 1, wherein the charged unit Z is positively or negatively charged.

6. The derivatization agent of claim 1, wherein the linker L2 comprises 1 to 10 C atoms and optionally one or more heteroatom(s).

7. The derivatization agent of claim 1, wherein the reactive group X is selected from the group consisting of a carbonyl reactive unit, a diene reactive unit, a hydroxyl reactive unit, an amino reactive unit, an imine reactive unit, a thiol reactive unit, a diol reactive unit, a phenol reactive unit, an epoxide reactive unit, a disulfide reactive unit, and an azido reactive unit.

8. A kit comprising the derivatization agent according to claim 1.

9. (canceled)

10. A conjugate of a derivatization agent according to claim 1 and an analyte, wherein the conjugate has the structure of formula (II)
C-L1-Z-(L2)ₚ-Xa-Ya-A (II)
wherein C, L1, L2, p, and Z are as defined in claim 1; Xa is a remainder of a reactive group X as defined in claim 1; A is the analyte and Ya is the remainder of a reactive group Y bound to the analyte A, which has reacted with the reactive group X of the derivatization agent thus forming a covalent bond between Xa and Ya.

11. The conjugate of claim 10, wherein the analyte is selected from the group consisting of a nucleic acid, an amino acid, a peptide, a protein, a metabolite, a hormone, a fatty acid, a lipid, a carbohydrate, a steroid, a ketosteroid, a secosteroid, a molecule characteristic of a certain modification of another molecule, and a substance that has been internalized by the organism and a metabolite of such a substance.

12. The conjugate of claim 10, wherein the reactive group Y is selected from the group consisting of a carbonyl group, a diene group, a hydroxyl group, an amine group, an imine group, a thiol group, a diol group, a phenolic group, an expoxid group, a disulfide group, and an azide group.

13. The conjugate of claim 10, wherein the conjugate has a molecular weight in the range of from ≥500 g/mol and/or a M⁺ peak in a mass spectrum of m/z≥500 and/or comprising a neutral loss unit C-L1-Z, which has a molecular weight of ≥300 g/mol and/or a peak in a mass spectrum of m/z≥300.

14. A method for the mass spectrometric determination of an analyte molecule, the method comprising the steps:
(a) providing an analyte of interest;
(b) providing a derivatization agent comprising a structure of formula (I) as defined in claim 1;
(c) reacting the analyte provided according to (a) with the derivatization agent provided according to (b), whereby a conjugate of the analyte and the derivatization agent is formed; and
(d) subjecting the conjugate formed in (c) to a mass spectrometric analysis.

15. The derivatization agent of claim 1, wherein the derivatisation agent is intended to be analyzed via laser desorption ionization mass spectrometry (LDI-MS).

16. The derivatization agent of claim 3, wherein the dotted line represents a single bond to the linker.

17. The method of claim 14, wherein the conjugate is covalently bound.

18. The method of claim 14, wherein the mass spectrometric analysis is selected from the group consisting of laser desorption ionization mass spectrometry (LDI-MS), (MA)LDI-MS, (MA)LDI-MS/MS, (SA)LDI-MS, and (SA)LDI-MS/MS.

19. The conjugate of claim 10, wherein the chromophore C has a structure of formula (C) 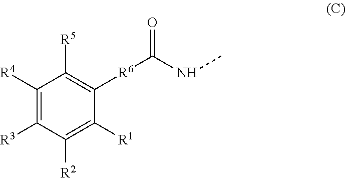
wherein R¹, R², R³, R⁴, R⁵ are independently selected from the group consisting of a hydrogen atom, a hydroxyl group, an NR^{x}R^{y} group, a C1 to C5 alkyl, a C5 to C10 (hetero)aryl, and an —O—C1 to C3 alkoxy group; wherein R^{x} and R^{y} are independently a hydrogen atom or a C1 to C5 alkyl group; wherein R⁶ is either none or is a —CR⁷═CR⁸— group; wherein R⁷ is a hydrogen atom or a C1 to C3 alkyl group; wherein R⁸ is selected from the group consisting of a hydrogen atom, a C1 to C5 alkyl group, and an electron withdrawing group; and wherein the dotted line represents a bond to the linker.
